(19) United States
(12) Patent Application Publication
(10) Pub. No.: US 2013/0111634 A1
(43) Pub. Date: May 2, 2013

(54) METHODS AND COMPOSITIONS FOR SILENCING GENES USING ARTIFICIAL MICRORNAS

(71) Applicant: E I Du Pont De Nemours And Company And Pion, Wilmington, DE (US)

(72) Inventors: Itzhak Kurek, San Francisco, CA (US); Brian McGonigle, Wilmington, DE (US); Genhai Zhu, San Jose, CA (US)

(73) Assignee: E I Du Pont De Nemours And Company And Pioneer Hi-Bred International, Wilmington, DE (US)

(21) Appl. No.: 13/661,237

(22) Filed: Oct. 26, 2012

Related U.S. Application Data
(60) Provisional application No. 61/552,700, filed on Oct. 28, 2011.

(51) Int. Cl. C12N 15/82; A01H 5/00; A01H 5/10; C12N 5/10

(52) U.S. Cl. 800/298; 435/320.1; 435/419; 435/415; 435/416; 435/412; 435/468

(57) ABSTRACT

Methods and compositions are provided that employ microRNA (miRNA) that, when expressed in a plant cell, is capable of reducing the level of mRNA of a target sequence (i.e. endogenous sequence) without reducing the level of mRNA of one or more closely related sequences. While miRNAs can be designed with specificity for a particular target sequence, the instant application demonstrates that a miRNA can specifically silence a target sequence without silencing a closely related sequence having high sequence identity to the target sequence. In certain embodiments, an endogenous target sequence can be suppressed with a recombinant miRNA expression construct without silencing a recombinant polynucleotide of interest having a sequence closely related to the target sequence. Such methods and compositions employ recombinant miRNA expression constructs which produce a 21-nt miRNA. Transgenic plant cells, plants and seeds incorporating miRNA expression constructs and recombinant polynucleotide constructs comprising polynucleotides of interest are also provided.

### CROSS REFERENCE TO RELATED APPLICATIONS

This application claims the benefit of U.S. Provisional Application No. 61/552,700, filed Oct. 28, 2011, the entire content of which is herein incorporated by reference.

### FIELD OF THE INVENTION

The field of the present invention relates generally to plant molecular biology. More specifically, it relates to constructs and methods to reduce the level of expression of a target sequence.

### BACKGROUND OF THE INVENTION

Biochemists and biotechnologists introduce altered (or shuffled) versions of genes into organisms with the intent to produce a desired phenotype. However, the desired outcome is often not obtained due to the presence of the endogenous gene product that still remains. Thus, there is a desire to replace endogenous genes with altered versions.

A variety of methods have been used in plants to overcome these problems; unfortunately, such methods have not proven sufficient for replacing endogenous genes with altered versions. For example, traditional RNAi silencing using long DS-RNA has not proven effective because the homology between the endogenous and introduced genes results in silencing of both genes. DS-RNA that targets the promoters of the endogenous genes has shown some promise, but the efficacy of silencing is frequently not sufficient and because the promoter is silenced it is impossible to use the endogenous promoter to express the introduced gene. Thus, methods and compositions are needed in plants to allow an altered version of a gene that encodes a protein with improved characteristics to be expressed while eliminating or reducing the expression of the endogenous version of the gene.

### BRIEF SUMMARY OF THE INVENTION

Methods and compositions are provided that employ a microRNA (miRNA) that, when expressed in a plant cell, is capable of reducing the level of mRNA of a target sequence (i.e. an endogenous sequence) without reducing the level of mRNA of one or more closely related sequences. While miRNAs can be designed with specificity for a particular target sequence, the instant application demonstrates that a miRNA can specifically silence a target sequence without silencing a closely related sequence having high sequence identity to the target sequence. In certain embodiments, a target sequence (i.e. an endogenous sequence) can be suppressed with a recombinant miRNA expression construct without silencing a recombinant polynucleotide of interest having a sequence closely related to the target sequence. Such methods and compositions employ recombinant miRNA expression constructs which produce a 21-nt miRNA. Transgenic plant cells, plants and seeds incorporating miRNA expression constructs and recombinant polynucleotide constructs comprising polynucleotides of interest are also provided.

### BRIEF DESCRIPTION OF THE DRAWINGS AND SEQUENCES

FIG. 1 is a diagram of the PHP39309 plasmid.

FIG. 2 is a diagram of the PHP39307 plasmid.

FIG. 3 is a diagram of the PHP39308 plasmid.

FIG. 4 is a diagram of the PHP40973 plasmid.

FIG. 5 is a diagram of the PHP38464 plasmid.

FIG. 6 is a diagram of the PHP38463 plasmid.

FIG. 7 is a diagram of the PHP38465 plasmid.

FIG. 8 is a diagram of the PHP38462 plasmid.

The sequence descriptions and Sequence Listing attached hereto comply with the rules governing nucleotide and/or amino acid sequence disclosures in patent applications as set forth in 37 C.F.R. §1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2):345-373 (1984) which are herein incorporated by reference. The symbols and format used for nucleotide and amino acid sequence data comply with the rules set forth in 37 C.F.R. §1.822.

SEQ ID NO:1 is the nucleotide sequence of the DNA corresponding to the amiRNA referred to herein as PEPC4A.

SEQ ID NO:2 is the nucleotide sequence of the DNA corresponding to the amiRNA referred to herein as PEPC4B.

SEQ ID NO:3 is the nucleotide sequence of the DNA corresponding to the artificial star sequence in the 396h-PEPC4A amiRNA precursor.

SEQ ID NO:4 is the nucleotide sequence of the DNA corresponding to the artificial star sequence in the 396h-PEPC4b amiRNA precursor.

SEQ ID NO:5 is the nucleotide sequence of the DNA corresponding to the artificial star sequence in the 169r-PEPC4A amiRNA precursor.

SEQ ID NO:6 is the nucleotide sequence of the amiRNA precursor 396h-PEPC4A.

SEQ ID NO:7 is the nucleotide sequence of the amiRNA precursor 396h-PEPC4B.

SEQ ID NO:8 is the nucleotide sequence of the amiRNA precursor 169r-PEPC4A.

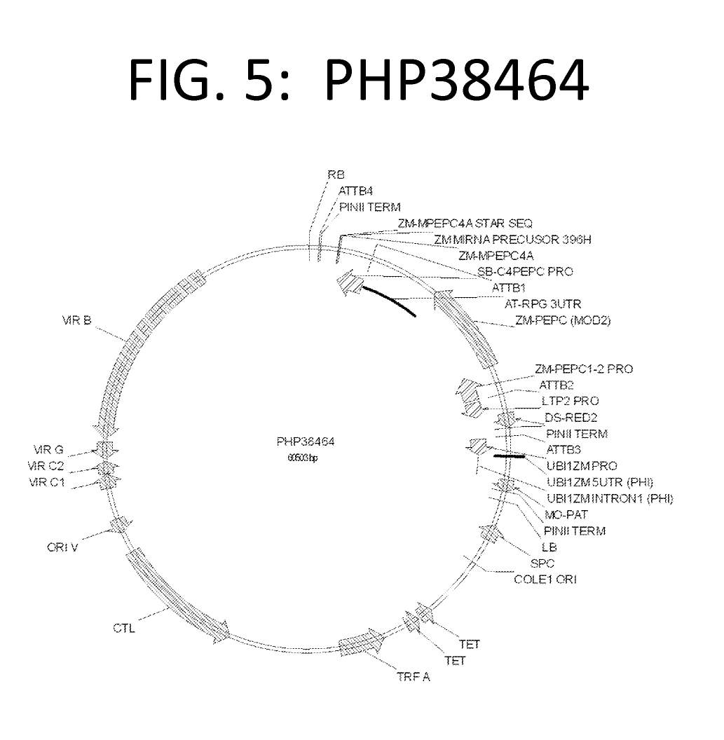

SEQ ID NO:9 is the nucleotide sequence of the PHP38464 plasmid (FIG. 5).

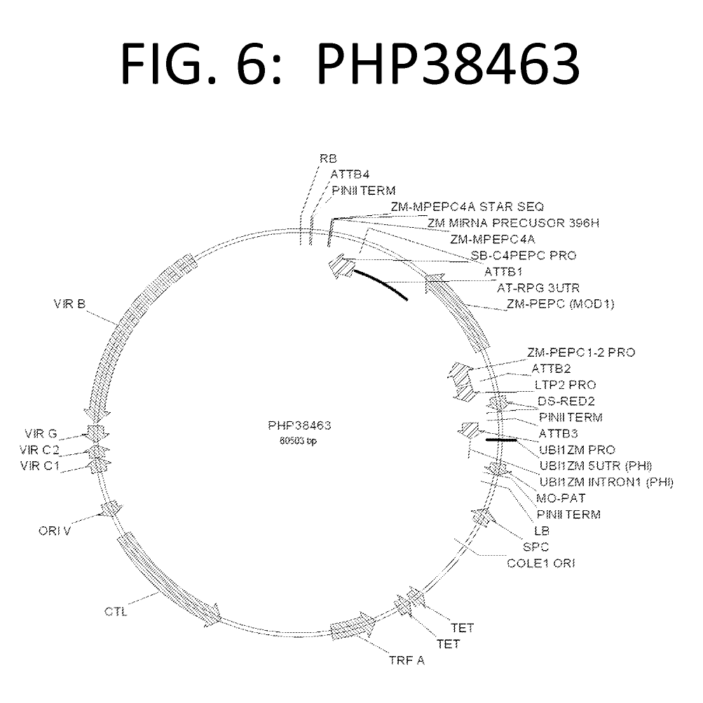

SEQ ID NO:10 is the nucleotide sequence of the PHP38463 plasmid (FIG. 6).

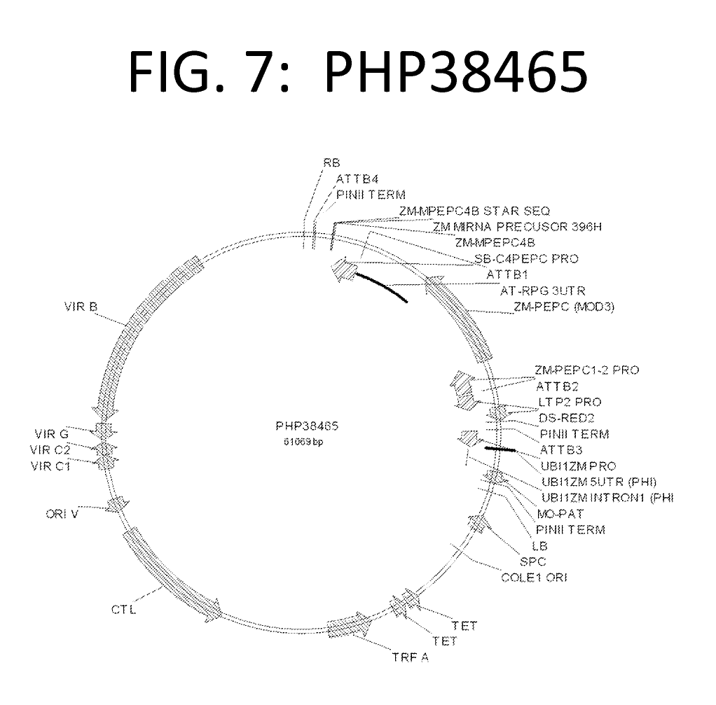

SEQ ID NO:11 is the nucleotide sequence of the PHP38465 plasmid (FIG. 7).

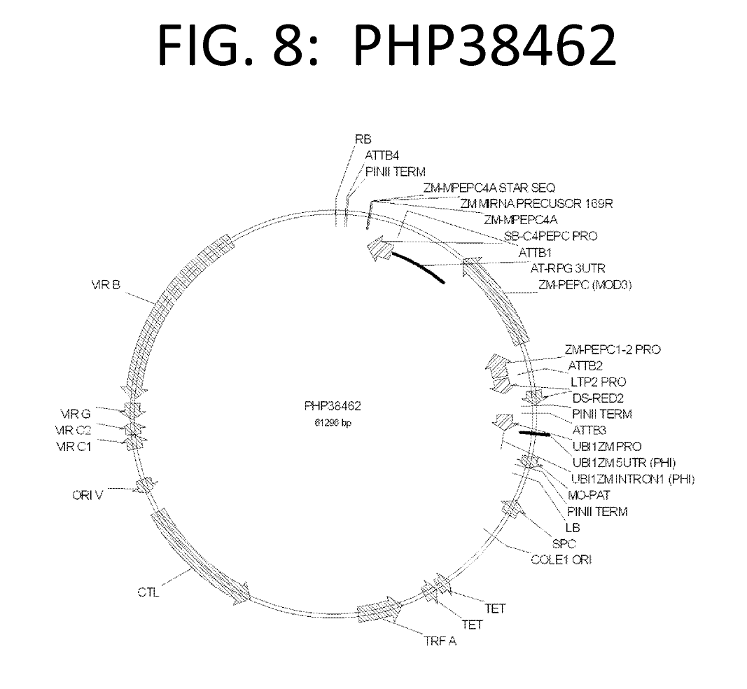

SEQ ID NO:12 is the nucleotide sequence of the PHP38462 plasmid (FIG. 8).

SEQ ID NO:13 is the nucleotide sequence of the DNA corresponding to the amiRNA referred to herein as RCA1a.

SEQ ID NO:14 is the nucleotide sequence of the DNA corresponding to the artificial star sequence in the 396h-RCA1a amiRNA precursor.

SEQ ID NO:15 is the nucleotide sequence of the DNA corresponding to the artificial star sequence in the 169r-RCA1a amiRNA precursor.

SEQ ID NO:16 is the nucleotide sequence of the amiRNA precursor 396h-RCA1a.

SEQ ID NO:17 is the nucleotide sequence of the amiRNA precursor 169r-RCA1a.

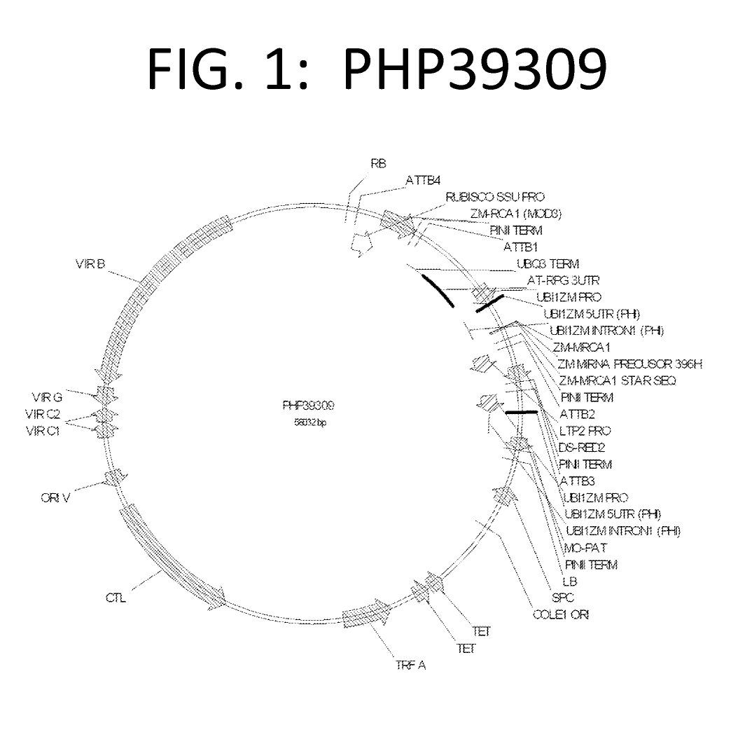

SEQ ID NO:18 is the nucleotide sequence of the PHP39309 plasmid (FIG. 1).

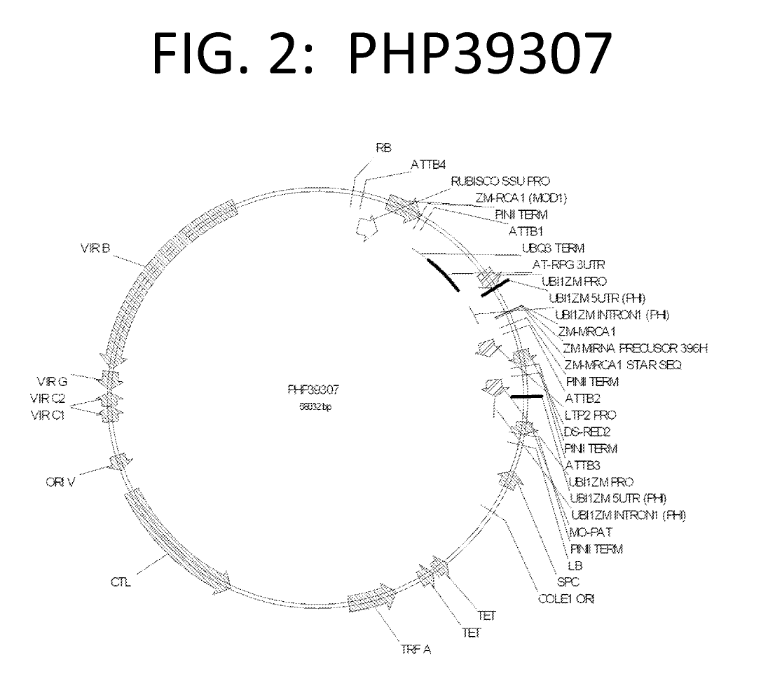

SEQ ID NO:19 is the nucleotide sequence of the PHP39307 plasmid (FIG. 2).

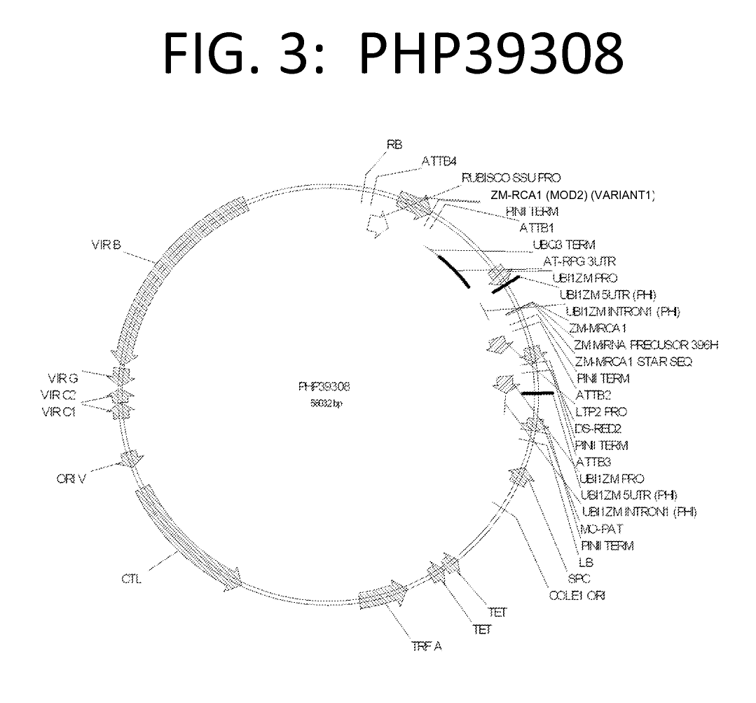

SEQ ID NO:20 is the nucleotide sequence of the PHP39308 plasmid (FIG. 3).

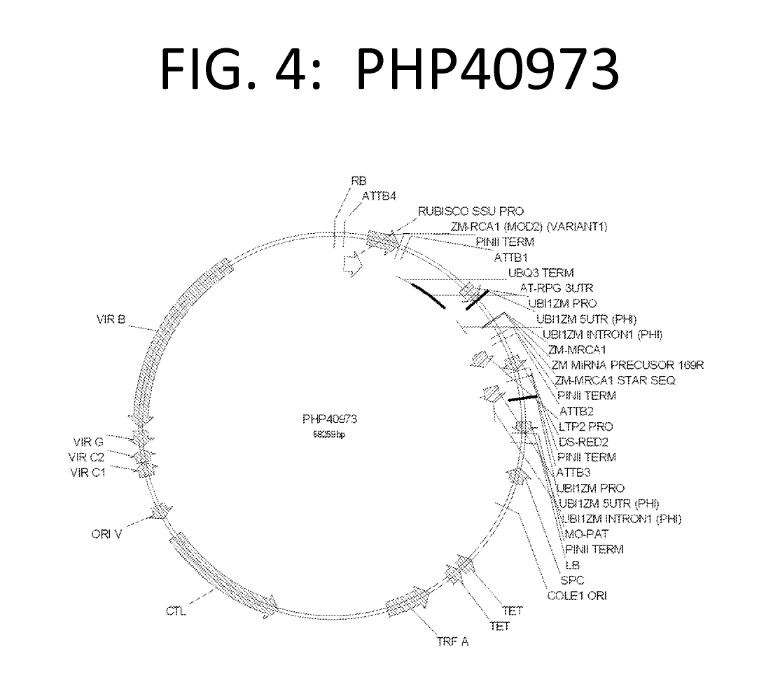

SEQ ID NO:21 is the nucleotide sequence of the PHP40973 plasmid (FIG. 4).

SEQ ID NO:22 is the nucleotide sequence of the Rubisco Activase 1 gene in maize (ZmRCA1; Genbank ID No. AF084478.3).

SEQ ID NO:23 is the nucleotide sequence of a shuffled version of ZmRCA1 herein referred to as ZmRCA1MOD1.

SEQ ID NO:24 is the nucleotide sequence of a shuffled version of ZmRCA1 herein referred to as ZmRCA1MOD2 (Variant 1).

SEQ ID NO:25 is the nucleotide sequence of a shuffled version of ZmRCA1 herein referred to as ZmRCA1MOD3.

SEQ ID NO:26 is the nucleotide sequence of the C4 form of phosphoenolpyruvate carboxylase (PEPC) in maize.

SEQ ID NO:27 is the nucleotide sequence of a shuffled version of PEPC herein referred to as ZmPEPCMOD2.

SEQ ID NO:28 is the nucleotide sequence of a shuffled version of PEPC herein referred to as ZmPEPCMOD3.

SEQ ID NO:29 is the nucleotide sequence of the C3 form of phosphoenolpyruvate carboxylase (PEPC) in maize (NCBI GI No. 429148).

SEQ ID NO:30 is the nucleotide sequence of the root form of phosphoenolpyruvate carboxylase (PEPC) in maize (NCBI GI No. 3132309).

SEQ ID NO:31 is the nucleotide sequence of a shuffled version of PEPC herein referred to as ZmPEPCMOD1.

SEQ ID NO:32 is the amino acid sequence of the protein encoded by SEQ ID NO:23 (ZmRCA1MOD1).

SEQ ID NO:33 is the amino acid sequence of the protein encoded by SEQ ID NO:24 (ZmRCA1MOD2 (Variant 1)).

SEQ ID NO:34 is the amino acid sequence of the protein encoded by SEQ ID NO:25 (ZmRCA1MOD3).

SEQ ID NO:35 is the amino acid sequence of the maize Rubisco Activase 1 protein (NCBI GI No. 162458161).

SEQ ID NO:36 is the amino acid sequence of the protein encoded by SEQ ID NO:31 (ZmPEPCMOD 1).

SEQ ID NO:37 is the amino acid sequence of the protein encoded by SEQ ID NO:27 (ZmPEPCMOD2).

SEQ ID NO:38 is the amino acid sequence of the protein encoded by SEQ ID NO:28 (ZmPEPCMOD3).

SEQ ID NO:39 is the amino acid sequence of the maize phosphoenolpyruvate carboxylase (PEPC) (NCBI GI No. 27764449).

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions are provided that employ a microRNA (miRNA) that, when expressed in a plant or in an appropriate cell, is capable of reducing the expression of a target sequence without reducing the expression of a closely related sequence. For example, the methods and compositions can allow for the expression of an improved version of a protein, while reducing the expression of a similar protein.

Such methods and compositions employ recombinant miRNA expression constructs. As used herein, a “recombinant miRNA expression construct” refers to a DNA construct which comprises a miRNA precursor backbone having a polynucleotide sequence encoding a miRNA and a star sequence. The recombinant miRNA expression constructs are designed such that the most abundant miRNA produced from the construct is a 21-nucleotide miRNA.

“microRNA” or “miRNA” refers to oligoribonucleic acid, generally of about 19 to about 24 nucleotides (nt) in length, which regulates expression of a polynucleotide comprising a target sequence. microRNAs are non-protein-coding RNAs and have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). miRNAs are derived, in plants, via dicer-like 1 processing of larger precursor polynucleotides. As discussed in further detail elsewhere herein, a miRNA can be an “artificial miRNA” or “amiRNA” which comprises a miRNA sequence that is synthetically designed to silence a target sequence.

Plant miRNAs regulate endogenous gene expression by recruiting silencing factors to complementary binding sites in target transcripts. microRNAs are initially transcribed as long polyadenylated RNAs and are processed to form a shorter sequence that has the capacity to form a stable hairpin and, when further processed by the siRNA machinery, release a miRNA. In plants, both processing steps are carried out by Dicer-like nucleases. miRNAs function by base-pairing to complementary RNA target sequences and trigger RNA cleavage of the target sequence by an RNA-induced silencing complex (RISC). microRNA molecules are highly efficient at inhibiting the expression of endogenous genes, and the RNA interference they induce is inherited by subsequent generations of plants.

### II. Compositions

A. Recombinant miRNA Expression Constructs Encoding 21-Nucleotide miRNAs

Recombinant miRNA expression constructs encoding a 21-nucleotide (21-nt) miRNA are provided herein. As used herein, a recombinant miRNA expression construct comprises a polynucleotide capable of being transcribed into an RNA sequence which is ultimately processed in the cell to form a miRNA. In some embodiments, the miRNA encoded by the recombinant miRNA expression construct is an artificial miRNA. Various modifications can be made to the recombinant miRNA expression construct to encode a miRNA. Such modifications are discussed in detail elsewhere herein.

In one embodiment, the recombinant miRNA expression construct comprises a miRNA precursor backbone having a heterologous miRNA and corresponding star sequence. As used herein, a “miRNA precursor backbone” is a polynucleotide that provides the backbone structure necessary to form a hairpin RNA structure which allows for the processing and ultimate formation of the miRNA. Thus, the miRNA precursor backbones are used as templates for expressing artificial miRNAs and their corresponding star sequence. Within the context of a recombinant miRNA expression construct, the miRNA precursor backbone comprises a DNA sequence having the heterologous miRNA and star sequences. When expressed as an RNA, the structure of the miRNA precursor backbone is such as to allow for the formation of a hairpin RNA structure that can be processed into a miRNA. In some embodiments, the miRNA precursor backbone comprises a genomic miRNA precursor sequence, wherein the sequence comprises a native precursor in which a heterologous miRNA and star sequence are inserted.

The miRNA precursor backbones can be from any source. In some embodiments, the miRNA precursor backbone is derived from a plant source. In some embodiments, the miRNA precursor backbone is from a monocot. In other embodiments, the miRNA precursor backbone is from a dicot. In further embodiments, the backbone is from maize or soybean. microRNA precursor backbones have been described previously. For example, US20090155910A1 discloses the following soybean miRNA precursor backbones: 156c, 159, 166b, 168c, 396b and 398b, and US20090155909A1 discloses the following maize miRNA precursor backbones: 159c, 164h, 168a, 169r, and 396h. Each of these references is incorporated by reference in their entirety. Non-limiting examples of miRNA precursor backbones disclosed herein include, for example, the miRNA ZM-169r precursor backbone or active variants thereof and the miRNA ZM-396h precursor backbone or active variants thereof. It is recognized that some modifications can be made to the miRNA precursor backbones provided herein, such that the nucleotide sequences maintain at least 60%, 70%, 75%, 80%, 85%, 90%, 95%, 96%, 97%, 98%, 99% or more sequence identity with the nucleotide sequence of the unmodified miRNA precursor backbone. Such variants of a miRNA precursor backbone retain miRNA precursor backbone activity and thereby continue to allow for the processing and ultimate formation of the miRNA.

When designing a recombinant miRNA expression construct to target a sequence of interest, the miRNA sequence of the backbone can be replaced with a heterologous miRNA designed to target any sequence of interest. In such instances, the corresponding star sequence in the recombinant miRNA expression construct will be altered such that the structure of the stem when folded remains the same as the endogenous structure. In such instances, both the star sequence and the miRNA sequence are heterologous to the miRNA precursor backbone.

Thus, in one embodiment, the miRNA precursor backbone can be altered to allow for efficient insertion of new miRNA and star sequences within the miRNA precursor backbone. In such instances, the miRNA segment and the star segment of the miRNA precursor backbone are replaced with the heterologous miRNA and the heterologous star sequence using a PCR technique and cloned into an expression plasmid to create the recombinant miRNA expression construct. It is recognized that there could be alterations to the position at which the heterologous miRNA and star sequences are inserted into the backbone. Detailed methods for inserting the miRNA and star sequence into the miRNA precursor backbone are described, for example, in US Patent Applications 20090155909A1 and US20090155910A1, herein incorporated by reference in their entirety.

In one embodiment, the miRNA precursor backbone comprises a first polynucleotide segment encoding a miRNA and a second polynucleotide segment encoding a star sequence, wherein the first and second polynucleotide segments are heterologous to the miRNA precursor backbone. As used herein, “heterologous” with respect to a sequence is intended to mean a sequence that originates from a foreign species, or, if from the same species, is substantially modified from its native form in composition and/or genomic locus by deliberate human intervention. For example, with respect to a nucleic acid, it can be a nucleic acid that originates from a foreign species, or is synthetically designed, or, if from the same species, is substantially modified from its native form in composition and/or genomic locus by deliberate human intervention. Thus, in the context of a recombinant miRNA expression construct, a heterologous miRNA and star sequence are not native to the miRNA precursor backbone. A recombinant miRNA expression construct comprising such a heterologous miRNA and star sequence can also be referred to as an “artificial” miRNA expression construct. Similarly, an “artificial” miRNA precursor backbone comprises a heterologous miRNA and star sequence with respect to the backbone.

The order of the miRNA and the star sequence within the recombinant miRNA expression construct can be altered. For example, in specific embodiments, the first polynucleotide segment comprising the miRNA segment of the recombinant miRNA expression construct is positioned 5′ to the second polynucleotide sequence comprising the star sequence. Alternatively, the second polynucleotide sequence comprising the star sequence can be positioned 5′ to the first polynucleotide sequence comprising the miRNA sequence in the recombinant miRNA expression construct.

As discussed above, the recombinant miRNA expression constructs are designed such that the most abundant form of miRNA produced from the recombinant miRNA expression construct is 21-nt in length. Such an expression construct will therefore comprise a first polynucleotide segment comprising the miRNA sequence and a second polynucleotide segment comprising the corresponding star sequence, wherein the star sequence and miRNA are 21-nt in length. In such instances, the star sequence and the miRNA sequence hybridize to each other. Such a structure results in a 21-nt miRNA being the most abundant form of miRNA produced.

As used herein, by “most abundant form” is meant the 21-nt miRNA representing the largest population of miRNAs produced from the recombinant miRNA expression construct. In other words, while the recombinant miRNA expression construct may produce miRNAs that are not 21-nt in length (i.e. 19-nt, 20-nt, 22-nt, etc.) the most abundant miRNA produced from the recombinant miRNA expression construct is 21-nt in length. Thus, the 21-nt miRNA represents at least 50%, 60%, 70%, 80%, 90%, 95% or 100% of the total miRNA population produced from the recombinant miRNA expression construct.

As used herein, a “star sequence” is the sequence within a miRNA precursor backbone that is complementary to the miRNA and forms a duplex with the miRNA to form the stem structure of a hairpin RNA. In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure. In still further embodiments, the star sequence comprises a sequence having 1, 2, 3, 4, 5 or more mismatches with the miRNA sequence and still has sufficient complementarity to form a double stranded structure with the miRNA sequence resulting in production of miRNA and suppression of the target sequence.

The most abundant miRNA produced from the recombinant miRNA expression construct is 21-nt in length and has sufficient sequence complementarity to a target sequence whose level of RNA is to be reduced. By “sufficient sequence complementarity” to the target sequence is meant that the complementarity is sufficient to allow the 21-nt miRNA to form a double stranded structure with the target sequence and reduce the level of expression of the target sequence. In specific embodiments, a miRNA having sufficient complementarity to the target sequence can share 100% sequence complementarity to the target sequence or it can share less than 100% sequence complementarity (i.e., at least 99%, 98%, 97%, 96%, 95%, 90%, 85%, 80%, 75%, 70% or less sequence complementarity) to the target sequence. In other embodiments, the miRNA can have 1, 2, 3, 4, 5 or up to 6 alterations or mismatches with the target sequence, so long as the 21-nt miRNA has sufficient complementarity to the target sequence to reduce the level of expression of the target sequence. Endogenous miRNAs with multiple mismatches with the target sequence have been reported. For example, see Schawb et al. (2005) Developmental Cell 8:517-27 and Cuperus et al. (2010) Nature Structural and Molecular Biology 17:997-1003, herein incorporated by reference in their entirety.

When designing a miRNA sequence and star sequence for the recombinant miRNA expression constructs disclosed herein, various design choices can be made. See, for example, Schwab R, et al. (2005) Dev Cell 8: 517-27. In non-limiting embodiments, the miRNA sequences disclosed herein can have a “U” at the 5′-end, a “C” or “G” at the 19^{th} nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the miRNA so that the sequence differs from the target sequence by one nucleotide.

B. Target Sequences

As used herein, “target sequence” refers to the sequence that the miRNA is designed to reduce and thus the expression of its RNA is to be modulated, e.g., reduced. The region of a target sequence of a gene of interest which is used to design the miRNA may be a portion of an open reading frame, 5′ or 3′ untranslated region, exon(s), intron(s), flanking region, etc. General categories of genes of interest include, for example, those genes involved in information, such as transcription factors, those involved in communication, such as kinases, and those involved in housekeeping, such as heat shock proteins. More specific categories, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like. The target sequence may be an endogenous sequence, or may be an introduced heterologous sequence. In a specific embodiment, the target sequence is a sequence endogenous to the plant cell. As used herein, an “endogenous” sequence is a native or naturally occurring sequence. When present within an organism, the endogenous sequence is native in that organism and present in its native genomic position.

Non-limiting examples of target sequences include, for example, members of the phosphoenolpyruvate carboxylase (PEPC) protein family or RUBISCO Activase 1.

PEPC is a member of the family of carboxy-lyases. PEPC influences the addition of bicarbonate to phosphoenolpyruvate to form oxaloacetate and is involved in carbon fixation and photosynthesis. In a non-limiting embodiment, the target sequence encodes a member of the phosphoenolpyruvate carboxylase protein family. Non-limiting examples of PEPC polynucleotide sequences from maize are set forth in SEQ ID NOs:26, 29, and 30. The DNA sequences corresponding to non-limiting examples of amiRNAs designed to reduce the level of mRNA of the PEPC having SEQ ID NO:26 are set forth in SEQ ID NOs:1 and 2.

RUBISCO, Ribulose-1,5-bisphophate carboxylase oxygenase, catalyzes the carboxylation or oxygenization of ribulose-1,5-bisphosphate with carbon dioxide or oxygen, which is a major rate-limiting step in photosynthesis. RUBISCO Activase is a member of the AAA⁺ super family and is involved in the activation of RUBISCO. RUBISCO Activase participates in the activation of RUBISCO by enhancing the removal of inhibitors from the active site of RUBISCO in an ATP-dependent manner. There are 2 isoforms of RUBISCO Activase, a 43 kDa and a 46 kDa isoform, formed by alternative splicing and differing only in the C-terminal region. In a non-limiting embodiment, the target sequence encodes RUBISCO Activase 1. A non-limiting example of a RUBISCO Activase 1 polynucleotide sequence from maize is set forth in SEQ ID NO:22. The DNA sequence corresponding to a non-limiting examples of an amiRNA designed to reduce the level of mRNA of RUBISCO Activase 1 is set forth in SEQ ID NO: 13.

The 21-nt miRNA produced from the recombinant miRNA expression construct is capable of reducing the level of mRNA of the target sequence without reducing the level of mRNA of a closely related recombinant polynucleotide of interest. Methods to assay for reduction in expression of mRNA include, for example, monitoring for a reduction in mRNA levels for the target sequence or monitoring for a change in phenotype. Various ways to assay for a reduction in the expression of a target sequence are discussed elsewhere herein. Thus, as disclosed herein, a single miRNA can silence a target sequence of interest, but not a closely related recombinant polynucleotide of interest.

As used herein, “reducing,” “suppression,” “silencing,” and “inhibition” are used interchangeably to denote the down-regulation of the level of expression of a product of a target sequence relative to its normal expression level in a wild type organism. By “reducing the level of RNA” is intended a reduction in expression by any statistically significant amount including, for example, a reduction of at least 10%, 15%, 20%, 25%, 30%, 35%, 40%, 45%, 50%, 55%, 60%, 65%, 70%, 75%, 80%, 85%, 90%, 95%, or 100% relative to the wild type expression level. As used herein, “without reducing the level of mRNA” or “not reduced” is intended any level of mRNA that is not reduced by any statistically significant amount relative to the mRNA level in the absence of expression of the recombinant miRNA expression construct, including, for example, a reduction in mRNA of about 9%, 8%, 7%, 6%, 5%, 4%, 3%, 2%, 1%, 0.5%, 0.1% or less. The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

C. Relationship Between the Target Sequence and the Closely Related Sequence

The miRNAs produced from the recombinant miRNA expression constructs disclosed herein can suppress a target sequence, but do not reduce the level of mRNA of a polynucleotide of interest having a sequence closely related to the target sequence. As used herein a “closely related” sequence is related to the target sequence such that the given nucleic acids of the closely related sequence and the target sequence share at least 50%, 55%, 60%, 65%, 70%, 75%, 80%, 85%, 90%, 91%, 92%, 93%, 94%, 95%, 96%, 97%, 98% or 99% sequence identity. The miRNAs produced from the recombinant miRNA expression constructs disclosed herein can suppress a target sequence such that the level of mRNA of at least 1, 2, 3, 4, 5 or more different sequences that are closely related to the target sequence are not reduced. In one embodiment, the target sequence is an endogenous sequence. In another embodiment, the closely related sequence is a recombinant polynucleotide of interest.

In a specific embodiment, the polynucleotide of interest is a shuffled variant of the target sequence. The term, “shuffling” or ‘shuffled” is used herein to indicate recombination between similar but non-identical polynucleotide sequences. As used herein, a “shuffled variant” is a new gene created by shuffling. Generally, more than one cycle of recombination is performed in shuffling methods. With such a procedure, one or more different genes of interest can be manipulated to create a new polynucleotide of interest possessing the desired properties. In this manner, libraries of recombinant polynucleotides are generated from a population of related sequence polynucleotides comprising sequence regions that have substantial sequence identity and can be homologously recombined in vitro and in vivo. For example, using this approach, sequence motifs encoding a domain of interest may be shuffled between the gene of interest and other known genes to obtain a new gene coding for a protein with an improved property of interest, such as Km in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

In one embodiment, the miRNA encoded by the recombinant miRNA expression construct corresponds to a complement of a region of the mRNA of the target sequence. The region of the mRNA of the target sequence can have 100% complementarity to the 21-nt miRNA, or the region of the mRNA of the target sequence can have at least 1, 2 or 3 non-complementary nucleotides to the 21-nt miRNA such that the miRNA reduces the level of mRNA of the target sequence but not the level of mRNA of a closely related polynucleotide of interest. As used herein, “complementary nucleotides”, “complementary sequence” or “complement” in reference to a sequence or region of nucleotides, are nucleotides that can form a double stranded structure. As such, “non-complementary” nucleotides are nucleotides that cannot form a double stranded structure. In further embodiments, the miRNA comprises at least 5, 6, 7, 8, 9, 10 or more non-complementary nucleotides to any given region across the length of the mRNA encoded by the polynucleotide of interest such that the miRNA reduces the level of mRNA of the target sequence but does not reduce the level of mRNA of the polynucleotide of interest.

In one embodiment, a first element comprising a recombinant expression construct comprising a polynucleotide of interest and a second element comprising a recombinant miRNA expression construct are present on the same polynucleotide construct. In such cases, the first element and the second element are integrated into the genome of a plant cell on the same construct. Further, the first and second elements can be operably linked to the same promoter. Alternatively, the first element and the second element can be present on separate polynucleotide constructs and are integrated into the genome of a plant cell on different polynucleotide constructs. In such cases, the first element comprises a first promoter operably linked to a sequence encoding a polynucleotide of interest and the second element comprises a second promoter operably linked to the recombinant miRNA expression construct.

D. Polynucleotides of Interest

The compositions further include various polynucleotides of interest. The polynucleotide of interest can be, but is not limited to, a native polynucleotide, a transgene, a shuffled variant of the target sequence, or any polynucleotide having a sequence closely related to the target sequence. In one embodiment, the miRNA, when expressed in a plant, reduces the level of mRNA of the target sequence without reducing the level of mRNA encoded by the polynucleotide of interest.

Various changes in phenotype are of interest, including modifying the fatty acid composition in a plant, altering the amino acid content of a plant, altering a plant's pathogen defense mechanism, altering a plant's tolerance to herbicides, and the like. These results can be achieved by providing expression of heterologous products (i.e. polynucleotides of interest). Alternatively, the results can be achieved by providing for a reduction of expression of one or more endogenous products, while at the same time providing expression of polynucleotides of interest in the plant. These changes result in a change in phenotype of the transformed plant.

Polynucleotides/polypeptides of interest include, but are not limited to, abiotic and biotic stress tolerance coding sequences, or sequences modifying plant traits such as yield, grain quality, nutrient content, starch quality and quantity, nitrogen fixation and/or utilization, and oil content and/or composition. More specific polynucleotides of interest include, but are not limited to, genes that improve crop yield, polypeptides that improve desirability of crops, genes encoding proteins conferring resistance to abiotic stress, such as drought, nitrogen, temperature, salinity, toxic metals or trace elements,

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Commercial traits can also be encoded on a polynucleotide of interest that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteria 170:5837-5847) facilitate expression of polyhyroxyalkanoates (PHAs).

Polynucleotides that improve crop yield include dwarfing genes, such as Rht1 and Rht2 (Peng et al. (1999) Nature 400:256-261), and those that increase plant growth, such as ammonium-inducible glutamate dehydrogenase. Polynucleotides that improve desirability of crops include, for example, those that allow plants to have reduced saturated fat content, those that boost the nutritional value of plants, and those that increase grain protein. Polynucleotides that improve salt tolerance are those that increase or allow plant growth in an environment of higher salinity than the native environment of the plant into which the salt-tolerant gene(s) has been introduced.

Polynucleotides/polypeptides that influence amino acid biosynthesis include, for example, anthranilate synthase (AS; EC 4.1.3.27) which catalyzes the first reaction branching from the aromatic amino acid pathway to the biosynthesis of tryptophan in plants, fungi, and bacteria. In plants, the chemical processes for the biosynthesis of tryptophan are compartmentalized in the chloroplast. See, for example, US Pub. 20080050506, herein incorporated by reference. Additional sequences of interest include Chorismate Pyruvate Lyase (CPL) which refers to a gene encoding an enzyme which catalyzes the conversion of chorismate to pyruvate and pHBA. The most well characterized CPL gene has been isolated from E. coli and bears the GenBank accession number M96268. See, U.S. Pat. No. 7,361,811, herein incorporated by reference.

In some embodiments, the polynucleotide of interest has a nucleotide sequence closely related to the nucleotide sequence of a member of the phosphoenolpyruvate carboxylase (PEPC) protein family. Non-limiting examples of polynucleotides of interest with closely related sequences to the PEPC gene set forth in SEQ ID NO:26 are represented by SEQ ID NOs:27, 28, and 31 or active variants and fragments thereof. In other embodiments, the polynucleotide of interest has a nucleotide sequence closely related to RUBISCO Activase 1. Non-limiting examples of polynucleotides of interest with closely related sequences to the RUBISCO Activase 1 gene set forth in SEQ ID NO:22 are represented by SEQ ID NOs:23, 24, and 25 or active variants and fragments thereof.

Active variants or fragments of polynucleotides/polypeptides of interest are also provided. Such active variants can comprise at least 65%, 70%, 75%, 80%, 85%, 90%, 91%, 92%, 93%, 94%, 95%, 96%, 97%, 98%, 99% or more sequence identity to the native polynucleotide/polypeptide of interest, wherein the active variants retain the biological activity of the native polynucleotide/polypeptide. Active variants or fragments of PEPC (i.e. SEQ ID NOs:27, 28, and 31 or active variants or fragments thereof) are provided herein such that they retain PEPC activity and thereby influence the formation of oxaloacetate. Any method known in the art can be used to assay for the activity of PEPC, including, but not limited to, measuring the formation of oxaloacetate in a sample in the presence of phosphoenolpyruvate, PEPC and carbon dioxide. Active variants and fragments of RUBISCO Activase 1 (i.e. SEQ ID NOs:23, 24, and 25 or active variants or fragments thereof) are also provided herein such that they retain RUBISCO Activase 1 activity and thereby induce RUBISCO activation. Any method known in the art can be used to assay for the activity of RUBISCO Activase, including, but not limited to, RUBISCO activation and ATP hydrolysis.

E. Polynucleotides

Compositions further include isolated or recombinant polynucleotides or polynucleotide constructs that encode the recombinant miRNA expression constructs, the various recombinant expression constructs that encode polynucleotides of interest, the various components of the recombinant miRNA expression constructs, along with the various products of the recombinant miRNA expression constructs that are processed into the miRNA. Exemplary components of the recombinant miRNA expression constructs include, for example, polynucleotides comprising miRNA precursor backbones, miRNA and star sequences, primers for generating the miRNAs and nucleotide sequences that encode the various RNA sequences. As used herein, “encodes” or “encoding” refers to a DNA sequence which can be processed to generate an RNA and/or polypeptide.

In one embodiment, a polynucleotide construct comprising a first element having a recombinant expression construct comprising a polynucleotide of interest and a second element comprising a recombinant miRNA expression construct is provided. In a specific embodiment, the first and second elements are operably linked to the same promoter.

The terms “polynucleotide,” “polynucleotide sequence,” “nucleic acid sequence,” and “nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides provided herein also encompass all forms of sequences including, but not limited to, single-stranded forms, double-stranded forms, hairpins, stem-and-loop structures, and the like.

The compositions provided herein can comprise an isolated or substantially purified polynucleotide. An “isolated” or “purified” polynucleotide is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived.

Further provided are recombinant polynucleotides comprising the polynucleotides of interest, the recombinant miRNA expression constructs and various components thereof. The terms “recombinant polynucleotide” and “recombinant DNA construct” are used interchangeably herein. A recombinant construct comprises an artificial or heterologous combination of nucleic acid sequences, e.g., regulatory and coding sequences that are not found together in nature. For example, a recombinant miRNA expression construct can comprise a miRNA precursor backbone having heterologous polynucleotides comprising the miRNA sequence and the star sequence and, thus the miRNA sequence and star sequence are not native to the miRNA precursor backbone. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

In specific embodiments, one or more of the expression constructs described herein can be provided in an expression cassette for expression in a plant or other organism or cell type of interest. The cassette can include 5′ and 3′ regulatory sequences operably linked to a polynucleotide provided herein. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of a recombinant polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a recombinant polynucleotide provided herein, and a transcriptional and translational termination region (i.e., termination region) functional in plants. The regulatory regions (i.e., promoters, transcriptional regulatory regions, and translational termination regions) and/or a recombinant polynucleotide provided herein may be native/analogous to the host cell or to each other. Alternatively, the regulatory regions and/or a recombinant polynucleotide provided herein may be heterologous to the host cell or to each other. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, the regulatory regions and/or a recombinant polynucleotide provided herein may be entirely synthetic.

The termination region may be native with the transcriptional initiation region, may be native with the operably linked recombinant polynucleotide of interest, may be native with the plant host, or may be derived from another source (i.e., foreign or heterologous) to the promoter, the recombinant polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al., (1991) Genes Dev. 5:141-149; Mogen et al., (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In preparing the expression cassettes, the various DNA fragments may be manipulated, so as to provide for the DNA sequences in the proper orientation. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the various expression constructs provided herein. The promoters can be selected based on the desired outcome. It is recognized that different applications can be enhanced by the use of different promoters in the recombinant expression constructs and/or the recombinant miRNA expression constructs to modulate the timing, location and/or level of expression of the polynucleotide of interest and/or the miRNA. Such recombinant expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, the expression constructs provided herein can be combined with constitutive, tissue-preferred, or other promoters for expression in plants. Examples of constitutive promoters include the cauliflower mosaic virus (CaMV) ³⁵S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT US01/22169).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat, A. et al. (1986) Plant Sci. 47:95-102; Reina, M. et al. Nucl. Acids Res. 18(21):6426; and Kloesgen, R. B. et al. (1986) Mol. Gen. Genet. 203:237-244. Promoters that express in the embryo, pericarp, and endosperm are disclosed in U.S. Pat. No. 6,225,529 and PCT publication WO 00/12733. The disclosures for each of these are incorporated herein by reference in their entirety.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of an expression construct within a particular plant tissue. Tissue-preferred promoters are known in the art. See, for example, Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and rubisco can also be used. See, for example, Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and roIB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengopta-Gopalen et al. (1988) PNAS 82:3320-3324.

The expression cassettes can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D) and sulfonylureas. Additional selectable markers include phenotypic markers such as beta-galactosidase and fluorescent proteins such as green fluorescent protein (GFP) (Su et al. (2004) Biotechnol. Bioeng. 85:610-9 and Fetter et al. (2004) Plant Cell 16:215-28), cyan fluorescent protein (CYP) (Bolte et al. (2004) J. Cell Science 117:943-54 and Kato et al. (2002) Plant Physiol. 129:913-42), and yellow fluorescent protein (PhiYFP™ from Evrogen; see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Nail. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the compositions presented herein.

F. Plants

Compositions comprising a transformed plant cell, a plant and a transgenic seed are further provided. In one embodiment, the transformed plant cell, plant or transgenic seed comprise a recombinant expression construct comprising a polynucleotide of interest having a sequence closely related to a target sequence (i.e an endogenous sequence) and a recombinant miRNA expression construct, wherein the recombinant miRNA expression construct encodes a miRNA consisting of 21-nucleotides and said miRNA when expressed in the plant cell reduces the level of mRNA of the target sequence (i.e. an endogenous sequence) without reducing the level of mRNA of the polynucleotide of interest.

It is recognized that the miRNA encoded by the recombinant miRNA expression construct can target any target sequence. In non-limiting embodiments, the target sequence encodes a member of the phosphoenolpyruvate carboxylase protein family or RUBISCO Activase 1. Any of the various miRNA precursor backbones, as described elsewhere herein, can be used in the recombinant miRNA expression constructs introduced into the plant cell, plant or seed. In addition, any of the various polynucleotides of interest discussed elsewhere herein (i.e. a native polynucleotide, a transgene, a shuffled variant of the target sequence, or any polynucleotide having a sequence closely related to the target sequence), can be used in the recombinant expression construct and expressed in the plant cell, plant or seed. In another embodiment, the encoded miRNA corresponds to a complement of a region of the mRNA of the target sequence such that the region has 3 or fewer non-complementary nucleotides to the 21-nt miRNA and the miRNA comprises 5 or more non-complementary nucleotides to any given region across the length of the mRNA encoded by the polynucleotide of interest. In specific embodiments, the complement of the region of mRNA of the target sequence can comprise 2 non-complementary nucleotides to the 21-nt miRNA, 1 non-complementary nucleotide to the 21-nt miRNA or has 100% sequence complementarity to the 21-nt-miRNA.

In some embodiments, the recombinant expression construct and the recombinant miRNA expression construct can be integrated into the genome of the plant cell on the same polynucleotide construct. Alternatively, the recombinant expression construct and the recombinant miRNA expression construct can be integrated into the genome of the plant cell on different polynucleotide constructs.

As used herein, “plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. The term “plant tissue” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in plant or in a plant organ, tissue or cell culture.

A transformed plant or transformed plant cell provided herein is one in which genetic alteration, such as transformation, has been affected as to a gene of interest, or is a plant or plant cell which is descended from a plant or cell so altered and which comprises the alteration. A “transgene” is a gene that has been introduced into the genome by a transformation procedure. Accordingly, a “transgenic plant” is a plant that contains a transgene, whether the transgene was introduced into that particular plant by transformation or by breeding; thus, descendants of an originally-transformed plant are encompassed by the definition. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which does not express the miRNA and/or a construct which does not express the polynucleotide of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the miRNA; or (e) the subject plant or plant cell itself, under conditions in which the recombinant miRNA expression construct and/or the recombinant expression construct comprising a polynucleotide of interest is not expressed.

Plant cells that have been transformed to have a recombinant expression construct and/or a recombinant miRNA expression construct provided herein can be grown into whole plants. The regeneration, development, and cultivation of plants from single plant protoplast transformants or from various transformed explants is well known in the art. See, for example, McCormick et al. (1986) Plant Cell Reports 5:81-84; Weissbach and Weissbach, In: Methods for Plant Molecular Biology, (Eds.), Academic Press, Inc. San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the compositions presented herein provide transformed seed (also referred to as “transgenic seed”) having a polynucleotide provided herein, for example, a recombinant miRNA expression construct, stably incorporated into their genome.

The recombinant expression constructs and recombinant miRNA expression constructs provided herein may be used for transformation of any plant species, including, but not limited to, monocots (e.g., maize, sugarcane, wheat, rice, barley, sorghum, or rye) and dicots (e.g., soybean, Brassica, sunflower, cotton, or alfalfa). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed herein include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants provided herein are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments soybean plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Depending on the target sequence, the transgenic plants, plant cells, or seeds expressing a recombinant expression construct and/or a recombinant miRNA expression construct provided herein may have a change in phenotype, including, but not limited to, an altered pathogen or insect defense mechanism, an increased resistance to one or more herbicides, an increased ability to withstand stressful environmental conditions, a modified ability to produce starch, a modified level of starch production, a modified oil content and/or composition, a modified carbohydrate content and/or composition, a modified fatty acid content and/or composition, a modified ability to utilize, partition and/or store nitrogen, and the like.

### III. Methods of Introducing

The methods provided herein comprise introducing into a plant cell, plant or seed a recombinant expression construct comprising a polynucleotide of interest and a recombinant miRNA expression construct encoding a 21-nt miRNA. Any of the various polynucleotides of interest, recombinant miRNA expression constructs or active variants and fragments thereof provided herein can be introduced into the plant cell, plant or seed.

In some embodiments, the recombinant miRNA expression construct and the recombinant expression construct comprising the polynucleotide of interest are introduced to the plant cell on the same polynucleotide construct. Alternatively, the recombinant miRNA expression construct and the recombinant expression construct are introduced into the plant cell on different polynucleotide constructs.

The methods provided herein do not depend on a particular method for introducing a sequence into the host cell, only that the polynucleotide gains access to the interior of a least one cell of the host. Methods for introducing polynucleotides into host cells (i.e. plants) are known in the art and include, but are not limited to, stable transformation methods, transient transformation methods, and virus-mediated methods.

The terms “introducing” and “introduced” are intended to mean providing a nucleic acid (e.g., a recombinant expression construct and/or recombinant miRNA expression construct or active variants or fragments thereof) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant expression construct and/or recombinant miRNA expression construct or active variants or fragments thereof) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

“Stable transformation” is intended to mean that the nucleotide construct introduced into a host (i.e., a plant) integrates into the genome of the plant and is capable of being inherited by the progeny thereof “Transient transformation” is intended to mean that a polynucleotide is introduced into the host (i.e., a plant) and expressed temporally.

Transformation protocols as well as protocols for introducing polynucleotide sequences into plants may vary depending on the type of plant or plant cell, i.e., monocot or dicot, targeted for transformation. Suitable methods of introducing polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the recombinant expression constructs and/or the recombinant miRNA expression constructs disclosed herein can be provided to a plant using a variety of transient transformation methods. Such transient transformation methods include, but are not limited to, the introduction of the recombinant expression constructs or the recombinant miRNA expression constructs or variants thereof directly into the plant. Such methods include, for example, microinjection or particle bombardment. See, for example, Crossway et al. (1986) Mol. Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the polynucleotides can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, recombinant expression constructs and recombinant miRNA expression constructs disclosed herein may be introduced into plants by contacting plants with a virus or viral nucleic acids. Generally, such methods involve incorporating a nucleotide construct provided herein within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the recombinant expression constructs and/or recombinant miRNA expression constructs provided herein can be contained in a transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The recombinant expression construct and/or the recombinant miRNA expression construct is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, transformed seed (also referred to as “transgenic seed”) having a recombinant expression construct and/or a recombinant miRNA expression construct disclosed herein, stably incorporated into their genome is provided.

### IV. Methods of Use

A method of reducing the level of mRNA of a target sequence in a plant cell, plant or seed by introducing into a plant cell, plant or seed a recombinant expression construct comprising a polynucleotide of interest and a recombinant miRNA expression construct encoding a 21-nt miRNA is provided. In such methods, the level of mRNA of the target sequence (i.e. an endogenous sequence) is reduced relative to the level of mRNA of the target sequence (i.e an endogenous sequence) in the absence of transcription of the recombinant miRNA expression construct and the level of mRNA of the polynucleotide of interest is not reduced relative to the level of mRNA of the polynucleotide of interest in the absence of transcription of the recombinant miRNA expression construct.

It is recognized that any miRNA that reduces the level of expression of the target sequence but does not reduce the level of mRNA of the polynucleotide of interest could be used in the methods provided herein. In addition, any of the various polynucleotides of interest disclosed herein (i.e. a native polynucleotide, a transgene, a shuffled variant of the target sequence, or any polynucleotide having a sequence closely related to the target sequence) can be used in the methods provided. In such methods, the encoded miRNA corresponds to a complement of a region of the mRNA of the target sequence wherein the region can have 3 or fewer non-complementary nucleotides to the 21-nt miRNA, 2 non-complementary nucleotides to the 21-nt miRNA, 1 non-complementary nucleotide to the 21-nt miRNA or 100% sequence complementarity to the 21-nt miRNA. In such cases, the miRNA comprises 5 or more non-complementary nucleotides to any given region across the length of the mRNA encoded by the polynucleotide of interest.

It is recognized that the miRNA encoded by the recombinant miRNA expression construct used in the methods can target any target sequence. In non-limiting embodiments, the target sequence encodes a member of the phosphoenolpyruvate carboxylase protein family or RUBISCO Activase 1. Any of the various miRNA precursor backbones, as described elsewhere herein, can be used in the recombinant miRNA expression constructs in the methods provided herein.

In the methods provided herein, the polynucleotide of interest and the recombinant miRNA expression construct can be present on the same polynucleotide construct or, alternatively, can be on different polynucleotide constructs. In specific embodiments, the recombinant expression construct comprises the polynucleotide of interest operably linked to a first promoter and the sequence encoding the recombinant miRNA expression construct is operably linked to a second promoter, wherein the first and second promoters are active in a plant. Alternatively, in some embodiments of the methods, the polynucleotide of interest of the recombinant expression construct and the miRNA expression construct are operably linked to the same promoter.

The methods provided herein can be used in any plant. In specific embodiments, the plant comprises a dicot or a monocot and in further embodiments, the dicot is soybean, Brassica, sunflower, cotton or alfalfa and the monocot is maize, sugarcane, wheat, rice, barley, sorghum or rye.

Any appropriate method can be used to assay for a reduced level of expression of a target sequence. For example, evaluation of reduced expression of a target nucleic acid in a plant or plant part, may be accomplished by a variety of means such as Northern analysis of mRNA expression, Western analysis of protein expression, or phenotypic analysis based on the function of the encoded proteins. In some embodiments, levels of other plant by-products such as oil can be analyzed as an indicator of a reduced level of expression of two or more sequences. Expression products of a target sequence can be detected in any of a variety of ways, depending upon the nature of the product (e.g., Western blot and enzyme assay). The level of expression of the polynucleotide of interest, whose level of mRNA is not reduced by the miRNA, can also be assayed by the above methods.

### V. Variants, Fragments and Sequence Comparisons

The methods and compositions provided herein employ a variety of different components. It is recognized throughout the description that some components can have active variants and fragments. Such components include, for example, any of the polynucleotides of interest, or any of the recombinant miRNA expression constructs or one of its components, such as the miRNA precursor backbone, the miRNA, or the star sequence (i.e. SEQ ID NOS: 1-21). Biological activity for each of these components is described elsewhere herein.

Active variants of the polynucleotides employed in the compositions and methods are further encompassed. For example, active variants of the polynucleotides of interest or any of the recombinant miRNA expression constructs or one of its components, such as the miRNA precursor backbone, the miRNA, or the star sequence are encompassed herein. “Variants” refer to substantially similar sequences. For polynucleotides, a variant comprises a deletion and/or addition of one or more nucleotides at one or more internal sites within the polynucleotide and/or a substitution of one or more nucleotides at one or more sites in the polynucleotide. Variants of the polynucleotides of interest, recombinant miRNA expression constructs, miRNA precursor backbones, miRNAs, and/or star sequences disclosed herein may retain activity of the polynucleotide of interest, recombinant miRNA expression construct, miRNA precursor backbone, miRNA, and/or star sequence as described in detail elsewhere herein. Variant polynucleotides can include synthetically derived polynucleotides, such as those generated, for example, by using site-directed mutagenesis. Generally, variants of a polynucleotide of interest, recombinant miRNA expression construct, miRNA precursor backbone, miRNA, and/or star sequence disclosed herein will have at least about 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Fragments of the polynucleotides of interest are also encompassed herein. By “fragment” is intended a portion of the polynucleotide or a portion of the amino acid sequence and hence protein encoded thereby. Fragments of a polynucleotide may encode protein fragments that retain the biological activity of the native protein. As used herein, a “native” polynucleotide or polypeptide comprises a naturally occurring nucleotide sequence or amino acid sequence, respectively. Thus, fragments of a polynucleotide may range from at least about 20 nucleotides, about 50 nucleotides, about 100 nucleotides, and up to the full-length polynucleotide. A fragment of a polynucleotide that encodes a biologically active portion of a protein employed in the methods or compositions will encode at least 15, 25, 30, 50, 100, 150, 200, or 250 contiguous amino acids, or up to the total number of amino acids present in a full-length protein. Alternatively, fragments of a polynucleotide that are useful as a hybridization probe or primer generally do not encode fragment proteins retaining biological activity. Thus, fragments of a nucleotide sequence may range from at least about 10, 20, 30, 40, 50, 60, 70, 80 nucleotides or up to the full length sequence.

A biologically active portion of a polypeptide can be prepared by isolating a portion of one of the polynucleotides encoding the portion of the polypeptide of interest and expressing the encoded portion of the protein (e.g., by recombinant expression in vitro), and assessing the activity of the portion of the polypeptide. For example, polynucleotides that encode fragments of a polypeptide of interest can comprise a nucleotide sequence comprising at least 16, 20, 50, 75, 100, 150, 200, 250, 300, 350, 400, 450, 500, 550, 600, 650, 700, 800, 900, 1,000, 1,100, 1,200, 1,300, or 1,400 nucleotides, or up to the number of nucleotides present in a nucleotide sequence employed in the methods and compositions provided herein.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence provided herein. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the specification as a whole.

Non-limiting examples of methods and compositions disclosed herein are as follows:

1. A polynucleotide construct comprising

(a) a first element comprising a recombinant expression construct comprising a polynucleotide of interest having at least 80% sequence identity to a target sequence; and,

(b) a second element comprising a recombinant miRNA expression construct,

wherein said recombinant miRNA expression construct encodes a miRNA consisting of 21 nucleotides (21-nt) and wherein said miRNA when expressed in a plant cell reduces the level of mRNA of the target sequence without reducing the level of mRNA of said first element.
2. The polynucleotide construct of embodiment 1, wherein said encoded miRNA corresponds to a complement of a region of the mRNA of the target sequence, wherein said region has 3 or fewer non-complementary nucleotides to said 21-nt miRNA; and, wherein said miRNA comprises 5 or more non-complementary nucleotides to any given region across the length of the mRNA encoded by the polynucleotide of interest.
3. The polynucleotide construct of embodiment 2, wherein said complement of a region of the mRNA of the target sequence comprises

(a) 2 non-complementary nucleotides to said 21-nt miRNA;

(b) 1 non-complementary nucleotide to said 21-nt miRNA; or

(c) 100% sequence complementarity to said 21-nt miRNA.

4. The polynucleotide construct of any one of embodiments 1-3, wherein the target sequence is endogenous to said plant cell.
5. The polynucleotide construct of any one of embodiments 1-4, wherein

(a) said first element comprises a first promoter operably linked to said sequence encoding the polynucleotide of interest; and

(b) said second element comprises a second promoter operably linked to said sequence encoding the recombinant miRNA expression construct;

wherein said first and second promoters are active in a plant.

6. The polynucleotide construct of any one of embodiments 1-4, wherein said first element and said second element are operably linked to the same promoter.
7. The polynucleotide construct of any one of embodiments 1-6, wherein said polynucleotide of interest is a shuffled variant of the target sequence.
8. The polynucleotide construct of embodiment 7, wherein said target sequence encodes a member of the phosphoenolpyruvate carboxylase protein family.
9. The polynucleotide construct of embodiment 7, wherein said target sequence encodes RUBISCO Activase 1.
10. A transformed plant cell comprising

(a) a recombinant expression construct comprising a polynucleotide of interest having at least 80% sequence identity when compared to an endogenous target sequence expressed in said plant cell; and,

(b) a recombinant miRNA expression construct capable of being transcribed into an RNA sequence in said plant cell,

wherein said recombinant miRNA expression construct encodes a miRNA consisting of 21 nucleotides (21-nt) and wherein said miRNA when expressed in said plant cell reduces the level of mRNA of said endogenous target sequence without reducing the level of mRNA of said polynucleotide of interest.

11. The transformed plant cell of embodiment 10, wherein said encoded miRNA corresponds to a complement of a region of the mRNA of the target sequence, wherein said region has 3 or fewer non-complementary nucleotides to said 21-nt miRNA; and,

wherein said miRNA comprises 5 or more non-complementary nucleotides to any given region across the length of the mRNA encoded by the polynucleotide of interest.

12. The transformed plant cell of embodiment 11, wherein said complement of a region of the mRNA of the target sequence comprises

(a) 2 non-complementary nucleotides to said 21-nt miRNA;

(b) 1 non-complementary nucleotide to said 21-nt miRNA; or

(c) 100% sequence complementarity to said 21-nt miRNA.

13. The transformed plant cell of any one of embodiments 10-12, wherein said recombinant expression construct comprising the polynucleotide of interest and said recombinant miRNA expression construct are integrated into the genome of the plant cell on the same polynucleotide construct.
14. The transformed plant cell of any one of embodiments 10-12, wherein said recombinant expression construct and said recombinant miRNA expression construct are integrated into the genome of the plant cell on different polynucleotide constructs.
15. The transformed plant cell of any one of embodiments 10-14, wherein said polynucleotide of interest is a shuffled variant of the target sequence.
16. The transformed plant cell of embodiment 15, wherein said target sequence encodes a member of the phosphoenolpyruvate carboxylase protein family.
17. The transformed plant cell of embodiment 15, wherein said target sequence encodes RUBISCO Activase 1.
18. A plant comprising the transformed plant cell of any one of embodiments 10-17.
19. A transgenic seed comprising the transformed plant cell of any one of embodiments 10-17.
20. The transformed plant cell of any one of embodiments 10-17, wherein said plant cell is from a dicot.
21. The transformed plant cell of embodiment 20, wherein said dicot is soybean, Brassica, sunflower, cotton, or alfalfa.
22. The transformed plant cell of any one of embodiments 10-17, wherein said plant cell is from a monocot.
23. The transformed plant cell of embodiment 22, wherein said monocot is maize, sugarcane, wheat, rice, barley, sorghum, or rye.
24. A method of reducing the level of mRNA of a target sequence in a plant cell comprising introducing into a plant cell

(a) a recombinant expression construct comprising a polynucleotide of interest having at least 80% sequence identity to an endogenous target sequence operably linked to a promoter active in the plant cell; and

(b) a recombinant miRNA expression construct, wherein said recombinant miRNA expression construct encodes a miRNA consisting of 21 nucleotides (21-nt);

wherein the level of mRNA of said endogenous target sequence is reduced relative to the level of mRNA of the endogenous target sequence in the absence of transcription of said recombinant miRNA expression construct, and wherein the level of mRNA of said polynucleotide of interest is not reduced relative to the level of mRNA of said polynucleotide of interest in the absence of transcription of said recombinant miRNA expression construct.

25. The method of embodiment 24, wherein said recombinant expression construct comprising said polynucleotide of interest and said recombinant miRNA expression construct are introduced into said plant cell on the same polynucleotide construct.
26. The method of embodiment 24, wherein said recombinant expression construct comprising said polynucleotide of interest and said recombinant miRNA expression construct are introduced into said plant cell on different polynucleotide constructs.
27. The method of any one of embodiments 24-26, wherein said encoded miRNA corresponds to a complement of a region of the mRNA of the target sequence, wherein said region has 3 or fewer non-complementary nucleotides to said 21-nt miRNA; and,

wherein said miRNA comprises 5 or more non-complementary nucleotides to any given region across the length of the mRNA encoded by the polynucleotide of interest.

28. The method of embodiment 27, wherein said complement of a region of the mRNA of the target sequence comprises

(a) 2 non-complementary nucleotides to said 21-nt miRNA;

(b) 1 non-complementary nucleotide to said 21-nt miRNA; or

(c) 100% sequence complementarity to said 21-nt miRNA.

29. The method of any one of embodiments 24-28, wherein

(a) said recombinant expression construct comprises said polynucleotide of interest operably linked to a first promoter; and

(b) said sequence encoding said recombinant miRNA expression construct is operably linked to a second promoter,

wherein said first and second promoters are active in a plant.

30. The method of any one of embodiments 24-28, wherein said recombinant expression construct and said recombinant miRNA expression construct are operably linked to the same promoter.
31. The method of any one of embodiments 24-30, wherein said polynucleotide of interest is a shuffled variant of the target sequence.
32. The method of embodiment 31, wherein said target sequence encodes a member of the phosphoenolpyruvate carboxylase protein family.
33. The method of embodiment 31, wherein said target sequence encodes RUBISCO Activase 1.
34. The method of any one of embodiments 24-33, wherein said plant cell is from a dicot.
35. The method of embodiment 34, wherein said dicot is soybean, Brassica, sunflower, cotton, or alfalfa.
36. The method of any one of embodiments 24-33, wherein said plant cell is from a monocot.
37. The method of embodiment 36, wherein said monocot is maize, sugarcane, wheat, rice, barley, sorghum, or rye.

### EXPERIMENTAL

The following examples are offered to illustrate, but not to limit, the claimed invention. It is understood that the examples and embodiments described herein are for illustrative purposes only, and persons skilled in the art will recognize various reagents or parameters that can be altered without departing from the spirit of the invention or the scope of the appended claims.

### Example 1

### Design of Artificial microRNA Sequences

Artificial microRNAs (amiRNAs) that would have the ability to silence the desired target genes are designed largely according to rules described in Schwab R, et al. (2005) Dev Cell 8: 517-27. To summarize, microRNA sequences are 21 nucleotides in length, have a “U” at their 5′-end, display 5′ instability relative to their star sequence (which is achieved by including a C or G at position 19), and have an “A” or a “U” at their 10th nucleotide. An additional requirement for artificial microRNA design is that the amiRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the amiRNA so that the sequence differs from the target sequence by one nucleotide.

### Example 2

### Design of Artificial Star Sequences

“Star sequences” are those that base pair with the amiRNA sequences, in the precursor RNA, to form imperfect stem structures. To form a perfect stem structure the star sequence would be the exact reverse complement of the amiRNA.

A precursor sequence (Zhang et al. (2006) FEBS Lett. 580(15):3753-62) can be folded using mfold (M. Zuker (2003) Nucleic Acids Res. 31: 3406-15; and D. H. Mathews, J. et al. (1999) J. Mol. Biol. 288: 911-940). The miRNA sequence is then replaced with the amiRNA sequence and the endogenous star sequence is replaced with the exact reverse complement of the amiRNA. Artificial star sequences can be designed by introducing changes in the star sequence such that the structure of the stem remains the same as the endogenous structure. The altered sequence is then folded with mfold, and the endogenous and altered structures are compared by eye. If necessary, further alterations to the artificial star sequence can be introduced to maintain structure.

### Example 3

### Conversion of Genomic microRNA Precursors to Artificial microRNA Precursors

Genomic miRNA precursor genes can be converted to amiRNAs using overlapping PCR and the resulting DNAs can be completely sequenced and then cloned into vectors for use in transformation.

Alternatively, amiRNAs can be synthesized commercially, for example by Codon Devices, (Cambridge, Mass.). The synthesized DNA is then cloned into a vector for use in transformation.

### Example 4

### Transformation of Maize

### A. Maize Particle-Mediated DNA Delivery

A DNA construct can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473-497, with 0.25 M sorbitol). The embryos on the high-osmotic medium are used as the bombardment target, and are left on this medium for an additional 18 hours after bombardment.

For particle bombardment, plasmid DNA (described above) is precipitated onto 1.8 mm tungsten particles using standard CaCl2-spermidine chemistry (see, for example, Klein et al., 1987, Nature 327:70-73). Each plate is bombarded once at 600 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, NY, pp 663-671.

Within 1-7 days after particle bombardment, the embryos are moved onto N6-based culture medium containing 3 mg/l of the selective agent bialaphos. Embryos, and later callus, are transferred to fresh selection plates every 2 weeks. The calli developing from the immature embryos are screened for the desired phenotype. After 6-8 weeks, transformed calli are recovered.

### B. Transformation of Maize Using Agrobacterium

Agrobacterium-mediated transformation of maize is performed essentially as described by Zhao et al., in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection and plant regeneration.

### 1. Immature Embryo Preparation:

Immature maize embryos are dissected from caryopses and placed in a 2 mL microtube containing 2 mL PHI-A medium.

### 2. Agrobacterium Infection and Co-Cultivation of Immature Embryos:

2.1 Infection Step:
PHI-A medium of (1) is removed with 1 mL micropipettor, and 1 mL of Agrobacterium suspension is added. The tube is gently inverted to mix. The mixture is incubated for 5 min at room temperature.

2.2 Co-culture Step:
The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

### Media for Plant Transformation:

1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4 D. After two weeks the tissue can be transferred to regeneration medium (Fromm et al., Bio/Technology 8:833 839 (1990)).

### Example 5

### Sequences and Vectors for the Silencing of Endogenous Phosphoenolpyruvate Carboxylase (PEPC) and Expression of Shuffled PEPC in Maize

Artificial miRNAs were designed to silence the C4 form of phosphoenolpyruvate carboxylase (PEPC) in maize (SEQ ID NO:26) and not the C3 (SEQ ID NO:29; NCBI GI No. 429148) nor root forms (SEQ ID NO:30; NCBI GI No. 3132309). One amiRNA referred to herein as PEPC4A was 5′-ucucugcagagccucaucgag-3′ (the DNA sequence corresponding to this amiRNA is represented by SEQ ID NO:1), and another, referred to herein as PEPC4B, was 5′-uucagaaacuccagaagccag-3′ (the DNA sequence corresponding to this amiRNA is represented by SEQ ID NO:2). The DNA sequences corresponding to the artificial star sequences that were used to silence phosphoenolpyruvate carboxylase are shown in Table 1.

TABLE 1
Artificial microRNA Star
Sequences for Silencing of PEPC

| | | SEQ |
|---|---|---|
| In amiRNA | Artificial Star | ID |
| precursor | Sequence | NO |
| (396h-PEPC4A) | ctcgatgaagctctgcagaga | 3 |
| (396h-PEPC4B) | ctggcttccggagtttctgaa | 4 |
| (169r-PEPC4A) | ttcgatgaggtctctgcagagc | 5 |

Genomic miRNA precursor genes were converted to amiRNA precursors using overlapping PCR (Example 3), and the resulting DNAs were completely sequenced. The following amiRNAs precursors were made:

TABLE 2
Artificial microRNA Precursor Sequences for Silencing of PEPC

| amiRNA | SEQ | Length |
|---|---|---|
| Precursor | ID NO | (nucs) |
| 396h-PEPC4A | 6 | 645 |
| 396h-PEPC4B | 7 | 645 |
| 169r-PEPC4A | 8 | 872 |

amiRNAs were then cloned using standard methods to produce vectors (Table 3) that contain the shuffled version of PEPC and the amiRNA targeted to the endogenous PEPC.

TABLE 3
Vectors for Silencing of Endogenous
PEPC and Expression of Shuffled PEPC

| | | Resulting | SEQ | |
|---|---|---|---|---|
| amiRNA | Shuffled PEPC | plasmid | ID NO | FIG |
| 396h-PEPC4A | ZmPEPC MOD2 | PHP38464 | 9 | 5 |
| | SEQ ID NO: 27 | | | |
| 396h-PEPC4A | ZmPEPC MOD1 | PHP38463 | 10 | 6 |
| | SEQ ID NO: 31 | | | |
| 396h-PEPC4B | ZmPEPC MOD3 | PHP38465 | 11 | 7 |
| | SEQ ID NO: 28 | | | |
| 169r-PEPC4A | ZmPEPC MOD3 | PHP38462 | 12 | 8 |
| | SEQ ID NO: 28 | | | |

### Example 6

### Sequences and Vectors for the Silencing of Endogenous Rubisco Activase 1 (RCA1) and Expression of Shuffled RCA in Maize

The artificial miRNA that was used to silence rubisco activase 1 in maize (ZmRCA1; SEQ ID NO:22; Genbank ID No. AF084478.3) was 5′-ucugcuucgucucguccaccu-3′ and is herein referred to as RCA1a (the DNA sequence corresponding to this amiRNA is represented by SEQ ID NO:13). The DNA sequences corresponding to the artificial star sequences that were used to silence rubisco activase are shown in Table 4.

TABLE 4
Artificial microRNA Star
Sequences for Silencing of RCA

| | | SEQ |
|---|---|---|
| In amiRNA | | ID |
| precursor | Star Sequence | NO |
| 396h-RCA1a | aggtggactagacgaagcaga | 14 |
| 169r-RCA1a | gggtggacgaagacgaagcagc | 15 |

Genomic miRNA precursor genes were converted to amiRNA precursors using overlapping PCR (Example 3), and the resulting DNAs were completely sequenced. The following amiRNAs precursors are made:

TABLE 5
Artificial microRNA Precursor Sequences
for the Silencing of RCA

| microRNA | SEQ | Length |
|---|---|---|
| Precursor | ID NO | (nucs) |
| 396h-RCA1a | 16 | 645 |
| 169r-RCA1a | 17 | 872 |

amiRNAs were then cloned using standard methods to produce vectors (Table 6) that contain the shuffled version of RCA and the amiRNA targeted to the endogenous RCA.

TABLE 6
Vectors for Silencing of Endogenous
RCA and Expression of Shuffled RCA

| | | Resulting | SEQ | |
|---|---|---|---|---|
| amiRNA | Shuffled RCA 1 | plasmid | ID NO | FIG |
| 396h-RCA1a | ZmRCA1 MOD3 | PHP39309 | 18 | 1 |
| | SEQ ID NO: 25 | | | |
| 396h-RCA1a | ZmRCA1 MOD1 | PHP39307 | 19 | 2 |
| | SEQ ID NO: 23 | | | |
| 396h-RCA1a | ZmRCA1 MOD2 | PHP39308 | 20 | 3 |
| | (VARIANT 1) | | | |
| | SEQ ID NO: 24 | | | |
| 169r-RCA1a | ZmRCA1 MOD2 | PHP40973 | 21 | 4 |
| | (VARIANT 1) | | | |
| | SEQ ID NO: 24 | | | |

### Example 7

### Quantification of RNA Expression Using qRT-PCR

Samples submitted for analysis are stored at −80 C until RNA isolation. RNA is isolated using the EZNA RNA kit (Omega Bio-Tek, Norcross, Calif., catalog #R1034-092) following manufacturer's conditions. The RNA is eluted in 60 of RNAse-free water and treated with 20 units of DNAse (Roche, Indianapolis, Ind.) following manufacturer's conditions. The DNAsed RNA is diluted with 4 volumes of 500 mM EDTA, pH 8 prior to inactivation of the DNAse by incubation at 65 C for 30 minutes. The absence of DNA in the final RNA prep had been determined in a previous experiment for the same type and amount of tissue, using QRTPCR reactions (see below) containing Taq polymerase enzyme only (no reverse transcriptase enzyme). The purity and absence of inhibition by the RNA in QRTPCR reactions had been determined in a previous experiment for the same type and amount of tissue, using the Agilent BioAnalyzer (purity) and QRTPCR analysis of serially diluted RNA, which showed the expected dose-response (absence of inhibition). A normalization control assay is used to account for well to well RNA concentration differences and is designed to the sequence of the corn RNA polymerase II large subunit transcript. The normalization control transcript is found to have a constant relationship to the concentration of RNA in similar samples, in a separate experiment. Real time QRTPCR assays are designed using Primer Express 3.0 (Applied Biosystems, Foster, Calif.). All Taqman™ probes are quenched with the minor groove binder (MGB). Primers were obtained from Integrated DNA Technologies (Coralville, Iowa) and MGB probes were obtained from Applied Biosystems.

For a comparative analysis of the RCA native transcript and the transcript produced from the shuffled RCA, an “allele discrimination” expression assay was developed. There are several sequence polymorphisms distinguishing the native RCA transcript from the introduced transgene, and a Taqman assay was designed to exploit these polymorphisms to confer the necessary specificity to the detection of each transcript. The RCA “allele discrimination” assay included a primer pair, which amplified both transcripts equally, and two probes: one probe (FAM-labeled) that only detects transgenic RCA and another probe (VIC-labeled) that only detects native RCA. The specificity of the assay was confirmed by testing non-transgenic samples, which showed only signal from the Vic-native RCA probe and no signal from the Fam-transgenic probe. In the RCA transcript analysis, the normalization control and RCA assays were run in separate reactions, and duplicates were analyzed.

For a comparative analysis of the PEPC native transcript and the transcript produced from the shuffled PEPC, two assays were designed, one to detect the native PEPC transcript and the other to detect the transcript produced from the shuffled PEPC. To detect the native PEPC, an assay was designed in the part of the native sequence not present in the transgenic construct. For analysis of the shuffled PEPC transcript, an assay to the 5 prime end of the UBQ3 terminator region was used. The PEPC and UBQ3 probes were both labeled with FAM. For the PEPC assays, the PEPC and normalization control assays were duplexed in the same reactions, and one replicate was analyzed.

The one step QRTPCR is performed according to manufacturer's suggestions using the SuperScriptlll Platinum One Step QRTPCR kit (Invitrogen, Carlsbad, Calif., catalog #11745-500). Ten microliter one-step QRTPCR reactions can contain 5 microliters of 2× master mix, 0.2 μl of 50×SSIII/Platinum Taq/RNAse OUT mixture, 8 picomoles of each primer and 0.8 picomoles of each probe, 4 microliters of RNA and RNAse-free water to volume. The Applied Biosytems 7900 instrument is used for real time thermal cycling, with conditions of: 3 minutes at 50 C (reverse transcription step), initial enzyme activation of 5 minutes at 95 C, and 40 cycles of 15 seconds at 95 C and 1 minute at 60 C (when fluorescence data is collected). Sequence Detection System version 2.2.1 is used for data collection and analysis. Calibrator samples are employed in all experiments in order to allow comparisons across experiments.

The calibrator RNA sample for each assay (RCA or PEPC) was a pool of samples obtained from transgenic plants that contain both native and shuffled transcripts. A non-transgenic maize RNA sample was tested in all assays (B73).

The cycle threshold (Ct) data was exported from SDS software to Microsoft Excel. The delta delta Ct method was validated and employed for relative expression calculations (User Bulletin#2, Applied Biosystems). The relative expression of each gene of interest can be described as “fold expression of the gene of interest, relative to its expression in the calibrator, normalized to the expression of the corn RNA polymerase II LSU gene”.

### Example 8

### Quantification of Protein Expression Using MS

### Sample Preparation

A total of 500 μL of T-CCLR buffer (100 mM KP pH 7.8, 1 mM EDTA, 7 mM BME, 1% Triton, 10% Glycerol and 1× Protease Inhibitor (CalBiochem Cat#539137, Protease Inhibitor Cocktail Set V. EDTA-Free)) is added per 10 leaf discs. Samples are mixed in a Spex Certiprep 2000 GenoGrinder at a setting of 1600 strokes/min for 1 min, centrifuged briefly. Grinding is repeated once and samples are then centrifuged (4° C., 3900 g) for 10 min. The supernatant is kept on ice, and total soluble proteins (TSPs) are measured with a Coomassie Protein Assay Reagent Kit (Pierce #23200). A total of 50 μL of supernatant is added to 110 μL of digestion buffer (50 mM ammonium bicarbonate (ABC); no adjustment of pH) in polymerase chain reaction (PCR) tubes. An appropriate amount of recombinant protein is spiked to blank matrix and used as standard curve. An appropriate amount of sequencing grade modified trypsin (Promega) is added (trypsin/TSP ratio ˜1:15) to all samples including standard curve. Samples are mixed briefly and spun in a microcentrifuge. Samples are then placed in a homemade sample holder fitted into a CEM Discover Proteomics System (Matthews, NC). Digestion is allowed to occur for 30 min (45° C., 50 W). After acidification with 10 μL of 10% (v/v) formic acid, samples are subject to LC-MS/MS analysis.

### LC-MS/MS

The LC-MS/MS system includes an AB Sciex 4000 Q-TRAP with a Turbo ion-spray source and Agilent 1100 LC. The autosampler temperature is kept at 6° C. during analysis. A total of 40 μL is injected onto an Aquasil, 100×2.1 mm, 3 μm, C18 column (ThermoFisher). LC is performed at a flow rate of 0.6 mL/min. Mobile phases consist of 0.1% formic acid (MPA) and 0.1% formic acid in acetonitrile (MPB). The total run time for each injection is ˜28 min. Below is the detailed gradient table:

| | Total | Flow | A | B |
|---|---|---|---|---|
| Step | Time(min) | Rate(μl/min) | (%) | (%) |
| 0 | 0.1 | 333 | 98 | 2 |
| 1 | 1 | 333 | 98 | 2 |
| 2 | 1.1 | 250 | 98 | 2 |
| 3 | 1.2 | 50 | 50 | 50 |
| 4 | 20 | 50 | 50 | 50 |
| 5 | 21 | 666 | 10 | 90 |
| 6 | 24.5 | 666 | 10 | 90 |
| 7 | 25.5 | 333 | 98 | 2 |
| 8 | 28 | 333 | 98 | 2 |

The mass spectrometer is operated in both multiple reaction monitoring (MRM) and linear ion-trap mode to select signature peptides. A complete list of MRM transitions is generated using MRM-initiated detection and sequencing (MIDAS) (AB Sciex) software for all tryptic peptides with an appropriate length (6-30 amino acids). The digested recombinant protein is analyzed using MRM-triggered information-dependent acquisition (IDA) to obtain both MRM chromatograms and MS/MS spectra, with the latter facilitating selection of the product ions with the highest sensitivity. The mass spectrometer is run in MRM mode at unit-mass resolution in both Q1 and Q3. The following electrospray ionization source parameters are used: dwell time, 200 ms for all MRM transitions; ion-spray voltage, 5500 V; ion source temperature, 555° C.; curtain gas (CUR), 20; both ion source gas 1 (GS1) and ion source gas 2 (GS2), 80; collision gas (CAD), high.

Chromatograms are integrated using AB Sciex software Analyst 1.4.2 with a Classic algorithm. Analyte peak areas are plotted against protein concentrations. A linear regression with 1/x2 (where x=concentration) weighting is used for calibration curve fitting.

The monitored MRM transitions were:

RCA WT (SEQ ID NO:35): 680.8/859.6, WVSETGVENIAR (doubly charged) and 388.2/575.3, EASDLIK (doubly charged)
RCA1 MOD1 (SEQ ID NO:32): 672.8/859.6, WVAETGVENIAR (doubly charged) RCA1 MOD2 (Variant 1) (SEQ ID NO:33): 380.2/559.6, EAADLIK (doubly charged) and 532.3/671.5, NFMSLPNIK (doubly charged)
RCA1 MOD3 (SEQ ID NO:34): 532.3/671.5, NFMSLPNIK (doubly charged)
PEPC WT (SEQ ID NO:39): 587.3/617.4, QEWLLSELR (doubly charged)
PEPC MOD1 (SEQ ID NO:36): 581.8/934.5, DILEGDPYLK (doubly charged) and 573.3/589.4, QEWLLSELK (doubly charged)
PEPC MOD2 (SEQ ID NO:37): 696.9/738.4, 696.9/851.5, VTLDLLEMIFAK (doubly charged)
PEPC MOD3 (SEQ ID NO:38): 540.3/879.5, LSAAWQLYK (doubly charged) and 573.3/589.4, QEWLLSELK (doubly charged)

### Example 9

### Analysis of Plants Expressing Shuffled PEPCase

Maize embryos from cultivar PH17AW were transformed by Agrobacterium containing plasmids PHP38464, PHP38463, PHP38465, or PHP38462 according to the protocol set out in Example 4. Transformants were screened. Plants containing only a single copy of the transgene were grown in the greenhouse, and leaf samples were collected for analysis. Controls were non-transgenic wild type PH17AW plants grown from seed and collected at a similar developmental stage. One skilled in the art would know that there are many methods of examining expression including RNA blot analysis, quantitative reverse-transcriptase polymerase chain reaction (qRT-PCR), Western blot analysis, ELISA, and MS protein determination. Expression was examined herein using both qRT-PCR (Example 7) and MS protein determination (Example 8); the results are shown in Tables 7-10.

TABLE 7
PHP38462 Results

| | |
|---|---|
| MS - Protein (ppm) | qRTPCR - mRNA |

| | shuffled | WT | shuffled | WT |
|---|---|---|---|---|
| Event ID | PEPC | PEPC | PEPC | PEPC |
| 119797417 | 10,254 | 1,668 | 44.71 | 4.22 |
| 119797418 | 4,532 | 351 | 59.71 | 2.16 |
| 119797419 | 8,095 | 1,563 | 31.40 | 1.93 |
| 119797420 | 1,094 | 32,003 | 0.04 | 53.01 |
| 119797421 | 3,106 | 23,998 | 0.03 | 47.72 |
| 119797422 | 8,150 | 1,402 | 30.54 | 2.93 |
| 119797423 | 4,619 | 663 | 49.42 | 5.47 |
| 119797424 | 17,268 | 1,433 | 28.15 | 1.07 |
| 119797425 | 21,785 | 12,402 | low RNA | 7.43 |
| 119797426 | 6,754 | 1,351 | 25.14 | 1.58 |
| 119797427 | 4,637 | 15,757 | 62.51 | 60.96 |
| 119797428 | 13,746 | 50,328 | 33.66 | 44.36 |
| 119797429 | 14,554 | 3,792 | 27.69 | 2.36 |
| 119797430 | 6,902 | 585 | 27.25 | 3.20 |
| 119797431 | 23,336 | 3,507 | 47.41 | 2.48 |
| 119797432 | 9,977 | 5,154 | 0.11 | 42.60 |
| 119797433 | 25,615 | 4,195 | 62.32 | 4.28 |
| 119797434 | 8,550 | 1,605 | 31.50 | 1.51 |
| 119797435 | 52,462 | 6,170 | 38.11 | 2.97 |
| 119797436 | 9,333 | 10,933 | 29.98 | 2.92 |
| 119797437 | 1,324 | 34,623 | 0.03 | 34.91 |
| 119797438 | 17,259 | 2,543 | 35.76 | 2.01 |
| 119797440 | 6,798 | 1,078 | 22.51 | 1.69 |
| 119797441 | 12,727 | 2,982 | 34.48 | 4.31 |
| 119797442 | 23,529 | 4,448 | 36.96 | 2.88 |
| 119797443 | 10,150 | 2,559 | 14.42 | 2.42 |
| 119797444 | 587 | 35,264 | 0.04 | 33.17 |
| 119797445 | 13,332 | 2,317 | 18.11 | 0.90 |
| 100845286 | | | 0.00 | 43.67 |
| (control) | | | | |
| 106867160 | | | 0.00 | 63.49 |
| (control) | | | | |

TABLE 8
PHP38463 Results

| | |
|---|---|
| MS - Protein (ppm) | qRTPCR - mRNA |

| | shuffled | WT | shuffled | WT |
|---|---|---|---|---|
| Event ID | PEPC | PEPC | PEPC | PEPC |
| 119798029 | 27,510 | 1,890 | 12.69 | 1.92 |
| 119798030 | 23,419 | 2,488 | 18.24 | 1.28 |
| 119798031 | 20,227 | 3,075 | 17.62 | 1.79 |
| 119798032 | 22,828 | 83,452 | 51.82 | 55.55 |
| 119798033 | 36,170 | 2,805 | 17.28 | 1.08 |
| 119798034 | 13,826 | 1,157 | 2.38 | 1.65 |
| 119798035 | 28,290 | 2,331 | 9.47 | 1.85 |
| 119798036 | 42,977 | 2,955 | 15.76 | 2.31 |
| 119798037 | 34,297 | 3,039 | 16.73 | 1.90 |
| 119798038 | 319 | 133,008 | 0.01 | 33.52 |
| 119798039 | 155 | 92,636 | 0.01 | 48.03 |
| 119798040 | 135 | 135,853 | 0.01 | 30.31 |
| 119798041 | 19,636 | 2,126 | 5.64 | 1.73 |
| 119798042 | 31,682 | 2,640 | 15.32 | 1.82 |
| 119798043 | 226 | 115,269 | 0.03 | 46.54 |
| 119798044 | 218 | 138,264 | 0.00 | 33.75 |
| 119798045 | 76 | 125,198 | 0.01 | 35.57 |
| 119798046 | 12,390 | 1,934 | 2.16 | 1.40 |
| 119798048 | 36,583 | 3,634 | 19.80 | 1.77 |
| 119798050 | 17,939 | 1,107 | 2.34 | 1.11 |
| 119798052 | 112 | 138,101 | 0.01 | 39.61 |
| 119798053 | 32,201 | 2,644 | 15.71 | 1.96 |
| 119798054 | 632 | 4,080 | 0.06 | 1.28 |
| 119798055 | 26,011 | 2,101 | 23.96 | 2.63 |
| 119798056 | 35,620 | 2,824 | 15.99 | 1.54 |
| 119798057 | 35,570 | 3,374 | 0.07 | 39.69 |
| 100845286 | | | 0.00 | 43.67 |
| (control) | | | | |
| 106867160 | | | 0.00 | 63.49 |
| (control) | | | | |

TABLE 9
PHP38464 Results

| | |
|---|---|
| MS - Protein (ppm) | qRTPCR - mRNA |

| | shuffled | WT | shuffled | WT |
|---|---|---|---|---|
| Event ID | PEPC | PEPC | PEPC | PEPC |
| 119267265 | 31,949 | 4,987 | 43.81 | 1.35 |
| 119267266 | 5,476 | 2,743 | 23.02 | 1.56 |
| 119267267 | 11,993 | 1,377 | 15.95 | 1.52 |
| 119267268 | 12,182 | 5,474 | 14.82 | 1.37 |
| 119267270 | 10,083 | 5,948 | 18.56 | 1.18 |
| 119267271 | 10,264 | 4,140 | 2.62 | 0.96 |
| 119267272 | 23,739 | 1,648 | 65.60 | 1.54 |
| 119267273 | 32,186 | 1,005 | 111.14 | 1.31 |
| 119267274 | 15,683 | 786 | 35.62 | 0.99 |
| 119267275 | 4,422 | 1,537 | 6.99 | 0.04 |
| 119267276 | 114 | 117,661 | 0.02 | 41.12 |
| 119267277 | 41,468 | 393 | 356.50 | 2.06 |
| 119267278 | 29,272 | 513 | 97.80 | 1.20 |
| 119267279 | 15,768 | 495 | 89.61 | 1.36 |
| 119267280 | 25,557 | 2,319 | 63.17 | 1.47 |
| 119267281 | 6,319 | 2,783 | 35.47 | 2.01 |
| 119267282 | 30,773 | 382 | 135.05 | 1.87 |
| 119267283 | 9,543 | 2,920 | 16.73 | 2.04 |
| 119267284 | 9,307 | 3,234 | 16.67 | 1.62 |
| 119267285 | 15,216 | 1,183 | 34.13 | 1.19 |
| 119267286 | 19,515 | 940 | 59.59 | 1.55 |
| 106867080 | | | 0.02 | 38.66 |
| (control) | | | | |
| 106867040 | | | 0.05 | 36.49 |
| (control) | | | | |

TABLE 10
PHP38465 Results

| | |
|---|---|
| MS - Protein (ppm) | qRTPCR - mRNA |

| | shuffled | WT | shuffled | WT |
|---|---|---|---|---|
| Event ID | PEPC | PEPC | PEPC | PEPC |
| 119953227 | 7,104 | 34,062 | 0.07 | 1.21 |
| 119953228 | 30,339 | 2,628 | 11.20 | 0.57 |
| 119953229 | 68,105 | 16,012 | 87.80 | 1.42 |
| 119953231 | 28,439 | 10,741 | 9.61 | 0.90 |
| 119953233 | 65,102 | 23,277 | 10.55 | 1.52 |
| 119953235 | 30,407 | 95,025 | 21.69 | 23.37 |
| 119953236 | 29,227 | 4,527 | 9.36 | 1.51 |
| 119953239 | 34,173 | 3,579 | 19.70 | 1.94 |
| 119953240 | 40,880 | 3,250 | 30.12 | 1.29 |
| 119953241 | 42,368 | 2,122 | 22.87 | 0.49 |
| 119953243 | 1,034 | 153,782 | 0.01 | 37.39 |
| 119953244 | 29,368 | 96,405 | 24.83 | 35.17 |
| 119953248 | 26,235 | 6,280 | 2.18 | 0.74 |
| 119953249 | 41,250 | 8,719 | 14.32 | 0.77 |
| 119953250 | 919 | 187,849 | not in use | 34.21 |
| 119953251 | 39,292 | 50,204 | 8.10 | 1.13 |
| 119953252 | 29,881 | 12,320 | 7.10 | 1.16 |
| 119953253 | 34,794 | 12,290 | 8.13 | 0.79 |
| 119953254 | 3,557 | 93,827 | 0.05 | 35.65 |
| 119953255 | 62,188 | 39,002 | 29.05 | 1.96 |
| 119953256 | 26,072 | 32,058 | 4.05 | 0.75 |
| 106867080 | | | 0.02 | 38.66 |
| (control) | | | | |
| 106867040 | | | 0.05 | 36.49 |
| (control) | | | | |

Tables 7-10 present quantitative RT-PCR and mass spectrometry protein results showing that miRNA can reduce the level of expression of the of the endogenous PEPC gene while allowing the shuffled variant of PEPC to be expressed. For example, event 119797417 in Table 7 shows that the amount of shuffled PEPC protein is on the order of 10,254 ppm, while the amount of endogenous (WT) PEPC protein is 1,668 ppm. Moreover, the amount of shuffled PEPC mRNA is over 10-fold greater than the amount of endogenous (WT) PEPC mRNA, as assessed using qRT-PCR. Multiple events showed similar results, thereby proving that constructs of the disclosure can be used to silence an endogenous gene while expressing a similar gene.

### Example 10

### Analysis of Plants Expressing Shuffled RCA1

Maize embryos from cultivar PH17AW were transformed by Agrobacterium containing plasmids PHP39309, PHP39307, PHP39308, or PHP40973 according to the protocol set out in Example 4. Transformants were screened. Plants containing only a single copy of the transgene were grown in the greenhouse, and leaf samples were collected for analysis. Controls were non-transgenic wild type PH17AW plants grown from seed and collected at a similar developmental stage. One skilled in the art would know that there are many methods of examining expression including RNA blot analysis, quantitative reverse-transcriptase polymerase chain reaction (qRT-PCR), Western blot analysis, ELISA, and MS protein determination. Expression was examined herein using both qRT-PCR (Example 7) and MS protein determination (Example 8); the results are shown in Tables 11-14.

TABLE 11
PHP39307 Results

| | |
|---|---|
| MS - protein (ppm) | qRTPCR - mRNA |

| | shuffled | WT | shuffled | Wild type |
|---|---|---|---|---|
| Event ID | RCA | RCA | mean | mean |
| 120823656 | 0 | 1988 | 0.00 | 3.75 |
| 120823653 | 4515 | 71 | 3.20 | 0.08 |
| 120823659 | 7675 | 169 | 7.71 | 0.12 |
| 120823651 | 4253 | 154 | 3.31 | 0.10 |
| 120823649 | 4205 | 175 | 3.63 | 0.16 |
| 120823650 | 10548 | 342 | 9.04 | 0.08 |
| 120823660 | 11309 | 360 | 8.55 | 0.20 |
| 120823638 | 5261 | 255 | 5.74 | 0.36 |
| 120823647 | 0 | 2043 | 0.00 | 6.40 |
| 120823654 | 5056 | 587 | 4.46 | 0.45 |
| 120823648 | 4863 | 136 | 5.83 | 0.15 |
| 120823655 | 3508 | 122 | 2.69 | 0.05 |
| 120823646 | 4241 | 93 | 2.75 | 0.15 |
| 120823657 | 15637 | 430 | 12.81 | 0.04 |
| 120823641 | 5814 | 822 | 6.19 | 1.25 |
| 120823645 | 3190 | 838 | 3.94 | 0.16 |
| 120823642 | 2661 | 67 | 4.37 | 0.03 |
| 120823643 | 4925 | 278 | 6.60 | 0.53 |
| 119276294 | | | 0.00 | 4.90 |
| (control) | | | | |
| 119276454 | | | 0.00 | 4.06 |
| (control) | | | | |

TABLE 12
PHP39308 Results

| | |
|---|---|
| MS - protein (ppm) | qRTPCR - mRNA |

| | shuffled | WT | shuffled | Wild type |
|---|---|---|---|---|
| Event ID | RCA | RCA | mean | mean |
| 120823787 | 3439 | 3 | 4.45 | 0.11 |
| 120823785 | 116 | 2802 | 0.01 | 9.89 |
| 120823789 | 12182 | 339 | 8.86 | 0.22 |
| 120823786 | 128 | 2009 | 0.01 | 4.44 |
| 120823784 | 3875 | 0 | 5.51 | 0.04 |
| 120823788 | 6869 | 42 | 10.89 | 0.02 |
| 120823805 | 2402 | 22 | 4.26 | 0.06 |
| 120823809 | 6705 | 330 | 7.45 | 0.47 |
| 120823806 | 5850 | 410 | 7.90 | 0.36 |
| 120823804 | 5021 | 70 | 5.40 | 0.01 |
| 120823811 | 34 | 2604 | 0.01 | 8.55 |
| 120823796 | 3467 | 33 | 3.59 | 0.04 |
| 120823807 | 13372 | 241 | 13.11 | 0.14 |
| 120823803 | 11751 | 53 | 11.51 | 0.11 |
| 120823795 | 4490 | 157 | 3.95 | 0.18 |
| 120823810 | 4658 | 49 | 4.32 | 0.06 |
| 120823802 | 5298 | 131 | 4.42 | 0.02 |
| 120823798 | 6194 | 448 | 5.32 | 0.14 |
| 120823799 | 3305 | 68 | 1.98 | 0.11 |
| 120823794 | 4115 | 26 | 4.46 | 0.09 |
| 120823790 | 3622 | 34 | 4.23 | 0.02 |
| 120823801 | 4240 | 0 | 4.34 | 0.01 |
| 120823793 | 3098 | 105 | 3.82 | 0.05 |
| 120823797 | 3584 | 15 | 4.83 | 0.01 |
| 120823800 | 5478 | 175 | 7.46 | 0.03 |
| 120823791 | 8011 | 51 | 9.13 | 0.03 |
| 120823792 | 54 | 1764 | 0.01 | 5.31 |
| 119276294 | | | 0.00 | 4.90 |
| (control) | | | | |
| 119276454 | | | 0.00 | 4.06 |
| (control) | | | | |

TABLE 13
PHP39309 Results

| | |
|---|---|
| MS - protein (ppm) | qRTPCR - mRNA |

| | shuffled | WT | shuffled | Wild type |
|---|---|---|---|---|
| Event ID | RCA | RCA | mean | mean |
| 120587523 | 5470 | 319 | 3.81 | 0.03 |
| 120587527 | 14571 | 940 | 5.50 | 0.13 |
| 120587514 | 3446 | 254 | 5.01 | 0.04 |
| 120587526 | 5310 | 386 | 10.54 | 0.05 |
| 120587505 | 7166 | 457 | 6.96 | 0.08 |
| 120587525 | 7408 | 470 | 5.56 | 0.03 |
| 120587509 | 4567 | 315 | 9.90 | 0.04 |
| 120587529 | 11699 | 237 | 10.39 | 0.02 |
| 120587516 | 2480 | 208 | 2.76 | 0.06 |
| 120587528 | 5327 | 703 | 4.48 | 0.11 |
| 120587524 | 8561 | 414 | 7.92 | 0.03 |
| 120587530 | 6300 | 428 | 0.51 | 0.00 |
| 120587504 | 5376 | 572 | 2.39 | 0.09 |
| 120587510 | 4345 | 300 | 5.52 | 0.05 |
| 120587507 | 14680 | 342 | 11.15 | 0.09 |
| 120587513 | 13986 | 272 | 15.46 | 0.05 |
| 120587519 | 3926 | 195 | 6.44 | 0.03 |
| 120587508 | 4881 | 306 | 5.94 | 0.06 |
| 120587520 | 14260 | 481 | 7.02 | 0.07 |
| 120587522 | 6185 | 259 | 6.65 | 0.04 |
| 120587515 | 3750 | 124 | 8.64 | 0.02 |
| 120587521 | 2905 | 179 | 4.24 | 0.08 |
| 120587518 | 14027 | 954 | 3.05 | 0.04 |
| 120587517 | 13061 | 501 | 5.14 | 0.02 |
| 119276328 | | | 0.00 | 4.08 |
| (control) | | | | |
| 119276329 | | | 0.00 | 5.15 |
| (control) | | | | |

TABLE 14
PHP40973 Results

| | |
|---|---|
| MS - protein (ppm) | qRTPCR - mRNA |

| | shuffled | WT | shuffled | Wild type |
|---|---|---|---|---|
| Event ID | RCA | RCA | mean | mean |
| 121566508 | 1790 | 4485 | 3.74 | 2.64 |
| 121566507 | 6608 | 4996 | 3.29 | 2.67 |
| 121566510 | 5045 | 5074 | 3.16 | 4.13 |
| 121566509 | 3504 | 5098 | 4.39 | 4.99 |
| 121566512 | 3930 | 3519 | 3.48 | 2.80 |
| 121566503 | 4423 | 4760 | 4.19 | 4.51 |
| 121566513 | 96 | 3637 | 0.00 | 4.19 |
| 121566514 | 4374 | 5028 | 2.63 | 3.76 |
| 121566494 | 1574 | 5562 | 3.61 | 5.91 |
| 121566495 | 4771 | 4276 | 4.02 | 4.48 |
| 121566498 | 6699 | 5534 | 9.39 | 5.62 |
| 121566504 | 2652 | 4225 | 5.64 | 5.72 |
| 121566499 | 6478 | 4362 | 4.29 | 4.01 |
| 121566501 | 2645 | 4984 | 1.30 | 3.76 |
| 121566506 | 11672 | 3853 | 5.15 | 1.98 |
| 121566497 | 4794 | 4993 | 2.52 | 4.61 |
| 121566502 | 5530 | 4204 | 4.12 | 3.20 |
| 121566505 | 0 | 3869 | 0.01 | 3.30 |
| 119276313 | | | 0 | 2.58 |
| (control) | | | | |
| 121657374 | | | 0 | 3.45 |
| (control) | | | | |

Tables 11-14 present quantitative RT-PCR and mass spectrometry protein results showing that miRNA can reduce the level of expression of the endogenous RUBISCO Activase 1 gene while allowing expression of a shuffled variant of RUBISCO Activase 1. For example, event 120823653 in Table 11 shows that the amount of shuffled RCA protein is on the order of 4,515 ppm, while the amount of endogenous (WT) RCA protein is 71 ppm. Moreover, the amount of shuffled RCA mRNA is 40-fold greater than the amount of endogenous (WT) PEPC mRNA, as assessed using qRT-PCR. Multiple events showed similar results, thereby proving that constructs of the disclosure can be used to silence an endogenous gene while expressing a similar gene.

### Example 11

### Silencing of Endogenous Gene and Expression of Shuffled Version in Soybean

Artificial miRNAs and artificial star sequences can be designed (as described in Examples 1 and 2, respectively) to silence a gene of interest in soybean. Genomic miRNA precursor genes can then be converted to amiRNA precursors using overlapping PCR (Example 3), and the resulting DNAs can be completely sequenced. Artificial miRNAs can then be cloned using standard methods to produce vectors that contain the shuffled version of a gene of interest and the amiRNA targeted to the endogenous gene. Transformation can occur, for example, as described in Example 12, and qRT-PCR and MS analyses can be performed, for example, as described in Examples 7 and 8.

### Example 12

### Transformation of Soybean

### Culture Conditions:

Soybean embryogenic suspension cultures (cv. Jack) are maintained in 35 mL liquid medium SB196 (infra) on a rotary shaker, 150 rpm, 26° C. with cool white fluorescent lights on 16:8 hr day/night photoperiod at light intensity of 60-85 μE/m²/s. Cultures are subcultured every 7 days to 2 weeks by inoculating approximately 35 mg of tissue into 35 mL of fresh liquid SB196 (the preferred subculture interval is every 7 days).

Soybean embryogenic suspension cultures are transformed with soybean expression plasmids by the method of particle gun bombardment (Klein et al., Nature, 327:70 (1987)) using a DuPont Biolistic PDS1000/HE instrument (helium retrofit) for all transformations.

### Soybean Embryogenic Suspension Culture Initiation:

Soybean cultures are initiated twice each month with 5-7 days between each initiation. Pods with immature seeds from available soybean plants 45-55 days after planting are picked, removed from their shells and placed into a sterilized magenta box. The soybean seeds are sterilized by shaking them for 15 min in a 5% Clorox solution with 1 drop of ivory soap (i.e., 95 mL of autoclaved distilled water plus 5 mL Clorox and 1 drop of soap, mixed well). Seeds are rinsed using 2 1-liter bottles of sterile distilled water and those less than 4 mm are placed on individual microscope slides. The small end of the seed is cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB 196 liquid media for 7 days.

### Preparation of DNA for Bombardment:

Either an intact plasmid or a DNA plasmid fragment containing the genes of interest and the selectable marker gene are used for bombardment. Fragments from soybean expression plasmids are obtained by gel isolation of digested plasmids. The resulting DNA fragments are separated by gel electrophoresis on 1% SeaPlaque GTG agarose (BioWhitaker Molecular Applications) and the DNA fragments containing gene cassettes are cut from the agarose gel. DNA is purified from the agarose using the GELase digesting enzyme following the manufacturer's protocol.

A 50 μL, aliquot of sterile distilled water containing 3 mg of gold particles is added to 5 μL, of a 1 μg/μL DNA solution (either intact plasmid or DNA fragment prepared as described above), 50 μL, 2.5 M CaCl₂ and 20 μL, of 0.1 M spermidine. The mixture is shaken 3 min on level 3 of a vortex shaker and spun for 10 sec in a bench microfuge. After a wash with 400 μL, of 100% ethanol, the pellet is suspended by sonication in 40 μL, of 100% ethanol. DNA suspension (5 mL) is dispensed to each flying disk of the Biolistic PDS1000/HE instrument disk. Each 5 μL, aliquot contains approximately 0.375 mg gold particles per bombardment (i.e., per disk).

Tissue Preparation and Bombardment with DNA:

Approximately 150-200 mg of 7 day old embryonic suspension cultures is placed in an empty, sterile 60×15 mm petri dish and the dish is covered with plastic mesh. Tissue is bombarded 1 or 2 shots per plate with membrane rupture pressure set at 1100 PSI and the chamber is evacuated to a vacuum of 27-28 inches of mercury. Tissue is placed approximately 3.5 inches from the retaining/stopping screen.

### Selection of Transformed Embryos:

Transformed embryos are selected using hygromycin as the selectable marker. Specifically, following bombardment, the tissue is placed into fresh SB196 media and cultured as described above. Six days post-bombardment, the SB196 is exchanged with fresh SB196 containing 30 mg/L hygromycin. The selection media is refreshed weekly. Four to six weeks post-selection, green, transformed tissue is observed growing from untransformed, necrotic embryogenic clusters. Isolated, green tissue is removed and inoculated into multiwell plates to generate new, clonally propagated, transformed embryogenic suspension cultures.

### Embryo Maturation:

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 E/m²s. After this time embryo clusters are removed to a solid agar media, SB166, for 1-2 weeks. Clusters are then subcultured to medium SB103 for 3 weeks.

### Media Recipes:

### SB196

### FN Lite Liquid Proliferation Medium (Per Liter)

| | | |
|---|---|---|
| MS FeEDTA—100x Stock 1 | 10 | mL |
| MS Sulfate—100x Stock 2 | 10 | mL |
| FN Lite Halides—100x Stock 3 | 10 | mL |
| FN Lite P, B, Mo—100x Stock 4 | 10 | mL |
| B5 vitamins (1 mL/L) | 1.0 | mL |
| 2,4-D (10 mg/L final concentration) | 1.0 | mL |
| KNO₃ | 2.83 | gm |
| (NH₄)₂SO₄ | 0.463 | gm |
| asparagine | 1.0 | gm |
| sucrose (1%) | 10 | gm |
| pH 5.8 | | |

### FN Lite Stock Solutions

| Stock | | | | | |
|---|---|---|---|---|---|
| Number | | 1000 mL | | 500 mL | |
| 1 | MS Fe EDTA 100x Stock | | | | |
| | Na₂ EDTA* | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

*Add first, dissolve in dark bottle while stirring

### SB1 Solid Medium (Per Liter)

1 package MS salts (Gibco/BRL—Cat. No. 11117-066)

1 mL B5 vitamins 1000× stock

31.5 g sucrose

2 mL 2,4-D (20 mg/L final concentration)

pH 5.7

8 g TC agar

### SB166 Solid Medium (Per Liter)

1 package MS salts (Gibco/BRL—Cat. No. 11117-066)

1 mL B5 vitamins 1000× stock

60 g maltose

750 mg MgCl₂ hexahydrate

5 g activated charcoal

pH 5.7

2 g gelrite

### SB103 Solid Medium (Per Liter)

1 package MS salts (Gibco/BRL—Cat. No. 11117-066)

1 mL B5 vitamins 1000× stock

60 g maltose

750 mg MgCl₂ hexahydrate

pH 5.7

2 g gelrite

### SB 71-4 Solid Medium (Per Liter)

1 bottle Gamborg's B5 salts with sucrose (Gibco/BRL—Cat. No. 21153-036)

pH 5.7

5 g TC agar

### 2,4-D Stock

Obtain premade from Phytotech Cat. No. D 295—concentration 1 mg/mL

### B5 Vitamins Stock (Per 100 mL)

Store aliquots at −20° C.

10 g myo-inositol

100 mg nicotinic acid

100 mg pyridoxine HCl

1 g thiamine

If the solution does not dissolve quickly enough, apply a low level of heat via the hot stir plate.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

MEGA

That which is claimed:

1. A polynucleotide construct comprising
(a) a first element comprising a recombinant expression construct comprising a polynucleotide of interest having at least 80% sequence identity to a target sequence; and,
(b) a second element comprising a recombinant miRNA expression construct,
wherein said recombinant miRNA expression construct encodes a miRNA consisting of 21 nucleotides (21-nt) and wherein said miRNA when expressed in a plant cell reduces the level of mRNA of the target sequence without reducing the level of mRNA of said first element.

2. The polynucleotide construct of claim 1, wherein said encoded miRNA corresponds to a complement of a region of the mRNA of the target sequence, wherein said region has 3 or fewer non-complementary nucleotides to said 21-nt miRNA; and,
wherein said miRNA comprises 5 or more non-complementary nucleotides to any given region across the length of the mRNA encoded by the polynucleotide of interest.

3. The polynucleotide construct of claim 2, wherein said complement of a region of the mRNA of the target sequence comprises
(a) 2 non-complementary nucleotides to said 21-nt miRNA;
(b) 1 non-complementary nucleotide to said 21-nt miRNA; or
(c) 100% sequence complementarity to said 21-nt miRNA.

4. The polynucleotide construct of any one of claims 1-3, wherein the target sequence is endogenous to said plant cell.

5. The polynucleotide construct of any one of claims 1-4, wherein
(a) said first element comprises a first promoter operably linked to said sequence encoding the polynucleotide of interest; and
(b) said second element comprises a second promoter operably linked to said sequence encoding the recombinant miRNA expression construct;
wherein said first and second promoters are active in a plant.

6. The polynucleotide construct of any one of claims 1-4, wherein said first element and said second element are operably linked to the same promoter.

7. The polynucleotide construct of any one of claims 1-6, wherein said polynucleotide of interest is a shuffled variant of the target sequence.

8. The polynucleotide construct of claim 7, wherein said target sequence encodes a member of the phosphoenolpyruvate carboxylase protein family.

9. The polynucleotide construct of claim 7, wherein said target sequence encodes RUBISCO Activase 1.

10. A transformed plant cell comprising:
(a) a recombinant expression construct comprising a polynucleotide of interest having at least 80% sequence identity when compared to an endogenous target sequence expressed in said plant cell; and,
(b) a recombinant miRNA expression construct capable of being transcribed into an RNA sequence in said plant cell,
wherein said recombinant miRNA expression construct encodes a miRNA consisting of 21 nucleotides (21-nt) and wherein said miRNA when expressed in said plant cell reduces the level of mRNA of said endogenous target sequence without reducing the level of mRNA of said polynucleotide of interest.

11. The transformed plant cell of claim 10, wherein said encoded miRNA corresponds to a complement of a region of the mRNA of the target sequence, wherein said region has 3 or fewer non-complementary nucleotides to said 21-nt miRNA; and,
wherein said miRNA comprises 5 or more non-complementary nucleotides to any given region across the length of the mRNA encoded by the polynucleotide of interest.

12. The transformed plant cell of claim 11, wherein said complement of a region of the mRNA of the target sequence comprises
(a) 2 non-complementary nucleotides to said 21-nt miRNA;
(b) 1 non-complementary nucleotide to said 21-nt miRNA; or
(c) 100% sequence complementarity to said 21-nt miRNA.

13. The transformed plant cell of any one of claims 10-12, wherein said recombinant expression construct comprising the polynucleotide of interest and said recombinant miRNA expression construct are integrated into the genome of the plant cell on the same polynucleotide construct.

14. The transformed plant cell of any one of claims 10-12, wherein said recombinant expression construct and said recombinant miRNA expression construct are integrated into the genome of the plant cell on different polynucleotide constructs.

15. The transformed plant cell of any one of claims 10-14, wherein said polynucleotide of interest is a shuffled variant of the target sequence.

16. The transformed plant cell of claim 15, wherein said target sequence encodes a member of the phosphoenolpyruvate carboxylase protein family.

17. The transformed plant cell of claim 15, wherein said target sequence encodes RUBISCO Activase 1.

18. A plant comprising the transformed plant cell of any one of claims 10-17.

19. A transgenic seed comprising the transformed plant cell of any one of claims 10-17.

20. The transformed plant cell of any one of claims 10-17, wherein said plant cell is from a dicot.

21. The transformed plant cell of claim 20, wherein said dicot is soybean, Brassica, sunflower, cotton, or alfalfa.

22. The transformed plant cell of any one of claims 10-17, wherein said plant cell is from a monocot.

23. The transformed plant cell of claim 22, wherein said monocot is maize, sugarcane, wheat, rice, barley, sorghum, or rye.

24. A method of reducing the level of mRNA of a target sequence in a plant cell comprising introducing into a plant cell
(a) a recombinant expression construct comprising a polynucleotide of interest having at least 80% sequence identity to an endogenous target sequence operably linked to a promoter active in the plant cell; and
(b) a recombinant miRNA expression construct, wherein said recombinant miRNA expression construct encodes a miRNA consisting of 21 nucleotides (21-nt);
wherein the level of mRNA of said endogenous target sequence is reduced relative to the level of mRNA of the endogenous target sequence in the absence of transcription of said recombinant miRNA expression construct, and wherein the level of mRNA of said polynucleotide of interest is not reduced relative to the level of mRNA of said polynucleotide of interest in the absence of transcription of said recombinant miRNA expression construct.

25. The method of claim 24, wherein said recombinant expression construct comprising said polynucleotide of interest and said recombinant miRNA expression construct are introduced into said plant cell on the same polynucleotide construct.

26. The method of claim 24, wherein said recombinant expression construct comprising said polynucleotide of interest and said recombinant miRNA expression construct are introduced into said plant cell on different polynucleotide constructs.

27. The method of any one of claims 24-26, wherein said encoded miRNA corresponds to a complement of a region of the mRNA of the target sequence, wherein said region has 3 or fewer non-complementary nucleotides to said 21-nt miRNA; and,
wherein said miRNA comprises 5 or more non-complementary nucleotides to any given region across the length of the mRNA encoded by the polynucleotide of interest.

28. The method of claim 27, wherein said complement of a region of the mRNA of the target sequence comprises
(a) 2 non-complementary nucleotides to said 21-nt miRNA;
(b) 1 non-complementary nucleotide to said 21-nt miRNA; or
(c) 100% sequence complementarity to said 21-nt miRNA.

29. The method of any one of claims 24-28, wherein
(a) said recombinant expression construct comprises said polynucleotide of interest operably linked to a first promoter; and
(b) said sequence encoding said recombinant miRNA expression construct is operably linked to a second promoter,
wherein said first and second promoters are active in a plant.

30. The method of any one of claims 24-28, wherein said recombinant expression construct and said recombinant miRNA expression construct are operably linked to the same promoter.

31. The method of any one of claims 24-30, wherein said polynucleotide of interest is a shuffled variant of the target sequence.

32. The method of claim 31, wherein said target sequence encodes a member of the phosphoenolpyruvate carboxylase protein family.

33. The method of claim 31, wherein said target sequence encodes RUBISCO Activase 1.

34. The method of any one of claims 24-33, wherein said plant cell is from a dicot.

35. The method of claim 34, wherein said dicot is soybean, Brassica, sunflower, cotton, or alfalfa.

36. The method of any one of claims 24-33, wherein said plant cell is from a monocot.

37. The method of claim 36, wherein said monocot is maize, sugarcane, wheat, rice, barley, sorghum, or rye.
